SEQUENCE LISTING (31 sequences)

SEQ ID NO: 1 - protein, 106 aa, Rhipicephalus pulchellus
AEKSLDSDSSGEDYELWTQGCPFLVAENRTGFGTTVSCQHNCNGAIEKVPEGEPCYTIGEDGLGRMKLNLPYNCSLGECSGGVCVPNGRSDVCFKRTWEENNKAMA

SEQ ID NO: 2 - protein, 97 aa, Amblyomma cajennense
ENTQQEEEDYDYGTDTCPFPVLANKTNKAKFVGCHQKCNGGDQKLTDGTACYVVERKVWDRMTPMLWYSCPLGECKNGVCEDLRKKEECRKGNGEEK

SEQ ID NO: 3 - protein, 104 aa, Rhipicephalus pulchellus
VCEVSEQEGVGEDNATEDEDYEDFFKPVTCYFANSTVGPLRPPNCTVVCTNNTAWWNDTKSDGGHCYSEYRPEKRTHSREIYNCTIGVCGNGTCIANHTYADCW

SEQ ID NO: 4 - protein, 89 aa, Rhipicephalus sanguineus
NEDDSSDYYDASPMNCSSMSVNSTMGWLSMNCTMSCNGTTFPLSNSTHCFHSYTNLTVQSRMETMTYNCSVGTCSNGTCVENGTTTTCW

SEQ ID NO: 5 - protein, 64 aa, Ixodes ricinus
RSKQPTASQSSKNSIKAEFCDTNCTQGTNGAWSGCSEGCFCVHVGNNTKGRCMKLSSDYDYTTQ

SEQ ID NO: 6 - protein, 97 aa, Amblyomma cajennense
ENTQQEEQDYDYGTDTCPFPVLANKTNKAKFVGCHQKCNGGDQKLTDGTACYVVERKVWDRMTPMLWYECPLGECKNGVCEDLRKKEDCRKGNGEEK

SEQ ID NO: 7 - protein, 98 aa, Amblyomma cajennense
EDTGTEDDFDYGNTGCPFPVLGNYKSNMTKPVGCKNKCGSGYEVLNDTTPCYVIDQKVFNNMVPLRQYSKCPLGFCENGECKPNDQAEDCYKGREEQK

SEQ ID NO: 8 - protein, 108 aa, Amblyomma parvum
DEESEELGASTDVDYEELDANCTCPAPALTSTRNNKHYPLGCIYNCSSYNCTIPDGTPCYVLTLGEVKEHLQIGSTVPNCTCGLCRNGTCVSNGTVEECFAVEEIEET

SEQ ID NO: 9 - protein, 108 aa, Amblyomma cajennense
ENGEGTTQPDYDNSTDYYNYEDFKCTCPAPHLNNTNGTVMKPIGCYYTCNVTRCTAPDTYPCYNLTEHQAKNLTTSPTTLCAVGNCDHGICVPNGTKELCFKAPNLEE

SEQ ID NO: 10 - protein, 94 aa, Amblyomma parvum
HRHYDPNEPCLIAGLKTPRDALPAGCRYDCMIKKNQKLRDGLLCLDVPEKVVKRMVNYLNYSCPLGTCRKGICKRKHRNVRCQKYPVFYMSPPK

SEQ ID NO: 11 - protein, 133 aa, Amblyomma maculatum
EKDTPKNIPGCGDTGTTAAPPADNPKHFAVYTDKHGCTIKVIGTWMTKEDHSRLPVSLRRVRASSGRVMLPASCQKICNDTVKNFPEGTPCRLVTGDPIKGKNHIKDGCIRGYCSSGVCVSDKRNISCYVPPN

SEQ ID NO: 12 - protein, 143 aa, Amblyomma cajennense
DTIGGIPGCGDPATTSAPEDQPKHYAVRTDKNGCKVMVIGTWMTKEDHNLLPPYISKERAPSGKVQLPASCKKNCHGKLKNLPNGTPCREVFGDLRRRRKHIKDGCKVGKCQNGLCVSEERIISCYLPPNITDPRPTHGPLAE

SEQ ID NO: 13 - protein, 146 aa, Amblyomma triste
EKDTPKNIPGCEDAGTTAAPPADNPKHYAVYTDKNGCTFKVIGTWMTKEDHRRLPVSLRRVRASSGRVKLPASCQKICKHSVKKFPEGTPCRLVTRDPIKGKNHIKNGCIRGYCSSGVCVSDNRSISCYVLPNNTELTSPSGSFAE

SEQ ID NO: 14 - protein, 143 aa, Amblyomma cajennense
DIIGGIPGCGDPTTTSAPEVPPKHFAVRTDKDGCTLMIIGTWMTTEDHNRLPPDIGEKRAPYGRVQLPASCKKNCHGKVKNLPNGTPCREVFGDPRRGRKHIKNGCTVGKCQSGLCVTDKRIISCYIPPNITDPIPTHGPWAE

SEQ ID NO: 15 - protein, 117 aa, Amblyomma cajennense
EEAPGPAPGCGEPEPTPPKPRRHGIVTNVNSCNSTILVWNGKEFPALCKVRCPHKSYRVSDFEPCLKFTNRRFLQERKDETPYKCKLGFCRHGTCITSEHSRKVPCKVPADRLDPSE

SEQ ID NO: 16 - protein, 148 aa, Amblyomma parvum
GPPSIPGNESIPGCGDAGTTTAPEDNPKHYGTLTDKKGCTLPIIGTWMTAVDHQHLQGTRGERRGPTGKVNLPASCRKNCHGRQEILRDGIPCRKVVGNPKGSKKHLKSGCLRGKCLAGQCVNDGRRISCYVPRNITDTEPTPGLLAE

SEQ ID NO: 17 - protein, 146 aa, Amblyomma triste
QSEVGKNVPGCGDTETLEAPPQEKPPYYEYKDEEGCTQKVLESWFQAGERSTGRGQKKRGHGRRPSRVLRTVDCRKNCTVGITALPDGHLCLVPRGDPFTRGGAIKYGCYLGDCASGHCQHRYETVSCRLPAPDTTAKPYYVTPEK

SEQ ID NO: 18 - protein, 88 aa, Ixodes ricinus
GPDTKGDEESDENELFTVEYCGTNCTQLENGSWTPCSGNNGNCRCFHESDKTVGLCLSTEYTDFSEYPDPNSSEIIAAAPLPRERLIQ

SEQ ID NO: 19 - protein, 80 aa, Ixodes ricinus
ADDDNELFTVQYCGMNCTKDEGGTWTGCTGKKEGCKCYHESGKNYGLCLSTEYTDFSQYGNPSDSEIEAAKPKRSDTLSH

SEQ ID NO: 20 - protein, 91 aa, Amblyomma triste
EEPKDGYDYTEGCPFVVLGNGTHAKPAGCSHLCNGAPETLDDNMECYNVTEEVAKRMTPDIPYTCWLGWCSKGECKRDNRTEVCYRGSERE

SEQ ID NO: 21 - protein, 90 aa, Amblyomma maculatum
EEREDDNDYGGGCPFVVLGNGTHAKPAGCSHLCNGAPETLDNIECYNVTEEVAKRMTPDIPYTCWLGWCSKGECKRDNRTEVCYRGSERE

SEQ ID NO: 22 - protein, 89 aa, Amblyomma maculatum
EPKDDNDYGGGCPFVVLGNGTHAKPAGCSHLCNGAPETLDNIECYNVTEEVAKRMTPGIPYACWLGWCNKGECKRGNRTEVCYRGSEEE

SEQ ID NO: 23 - protein, 90 aa, Amblyomma triste
EAPKDDFEYDGGCPFVVLDNGTHVKPAGCSHLCNGAPETLDNIECYNVTEEVAKRMTPGIPYACWLGWCSKGECKRDNRTEVCYRGSEEE

SEQ ID NO: 24 - protein, 159 aa, Amblyomma parvum
KTDTKNAAGELPPKVAIPGCEDPATTKAPLPDDPRYYGVTIDKDGCQRKVLGSSQRQQRQVQNGRKPGRKGRGRRPVFVDLKLTVDCKRKCNGTYSQLPDGEPCLVCDGEPYGRHRTIKGGCYQGNCSSGQCHRGERKVNCYIPKNITNNVLNSVNLAE

SEQ ID NO: 25 - protein, 159 aa, Amblyomma parvum
KTDTKNAAGELPPKVVIPGCEDPATTKAPLPDEPPYYGITIDKDGCQRKVLGSSQRQQRKVQNGRKLDGKKRGRRPVFVDLELTVDCKRKCNGTYSQLPDGENCLVSDGYPYGRWGTIKGGCYQGNCSSGQCHRGEKKVNCYLPKNITNDVPKSLNLAE

SEQ ID NO: 26 - protein, 141 aa, Ixodes ricinus
ASLAKETEDTTLPARALVDSPDSDNCSSPQLPYFDELTYMPLGFLAVNCTKTCPVGKNGTVVNGNKCIVTWSILDVSTITVLVGSCKNGYCISDGSSECRNITLAGEDSQEEEEEAEEDEEEDDGDEEEDEEEDEEENDDD

SEQ ID NO: 27 - protein, 119 aa, Ixodes ricinus
GSTPSAMKTEDILKVLGSTSSLENHTDSSHCRYQELLDITKNIENAGFLAINCQRSCPNGKQTMVEGYGCIFKIKHATKRGKVKVKEGSCRKGACVRGSTRPPWRLLVLLGESKEEEFL

SEQ ID NO: 28 - protein, 136 aa, Ixodes ricinus
SDLCKMEAESSPFKLPQSSLLDAPDEEGCKYQLLFVEAEGPLVVNCTKDCPNGKIRTVVEGELCIAMVKTSSSGEATGLVGSCKRGSCVKKDDPCRTFTLSEEGDDDEEDEEEEEDEEEDEEEDEEEEEEDEEEED

SEQ ID NO: 29 - protein, 98 aa, Amblyomma americanum
RNHTEDNSTEYYDYEEARCACPARHLNNTNGTVLKLLGCHYFCNGTLCTAPDGYPCYNLTAQQVRTLTTYPNTSCAVGVCMKGTCVKNGTMEQCFKTP

SEQ ID NO: 30 - protein, 151 aa, Amblyomma americanum
RGGAASVPANASIPGCGDAQTTPAAPEDQPKHYVVYRDGNGCEVKIIGTWMTTEDYNCLPDTFRKNRAPHGKVKLPASCKKTCGNAVQNLKDGTPCRKVFGDLGRRRNLIKNGCLVGACQSGLCVSGNRTISCYIPPNSTDTRATPGSFAE

SEQ ID NO: 31 - protein, 140 aa, Ixodes ricinus
KEPEDTTLPPGALVDSPDSDNCSSPHLPYFDETTNMWMGFLAVNCTKKCPVGKHVTVVDGNKCIGTWSFLDELTITVLVGSCKDGFCETDGSSECRNITLAEEDSQEEEGAAAEEEDEEDEEDEEEEEAEEEREDDHDDA

(19) United States
(12) Patent Application Publication
(10) Pub. No.: US 2022/0175942 A1
(43) Pub. Date: Jun. 9, 2022

(54) EVASINS FOR USE IN THERAPY AND DIAGNOSTICS

(71) Applicant: OXFORD UNIVERSITY INNOVATION LIMITED, Oxford (GB)

(72) Inventor: Shoumo Bhattacharya, Oxford (GB)

(21) Appl. No.: 17/403,740

(22) Filed: Aug. 16, 2021

Related U.S. Application Data
(63) Continuation of application No. 16/081,573, filed on Aug. 31, 2018, now abandoned.

(30) Foreign Application Priority Data
Mar. 2, 2016 (GB) 1603631.1

(51) Int. Cl. A61K 47/68; C07K 14/435; C07K 14/715; C07K 14/705; A61K 47/64; A61K 38/17; A61K 38/19; C07K 14/52; C12N 15/62

(52) U.S. Cl. CPC A61K 47/68; C07K 14/43527; C07K 14/7158; C07K 14/705; A61K 38/00; A61K 38/1774; A61K 38/195; C07K 14/521; C12N 15/62; A61K 47/642

(57) ABSTRACT

The invention relates to novel Evasin polypeptides with novel chemokine-binding properties and their use in inhibiting chemokines or detecting chemokine expression and inflammation.

### FIELD OF THE INVENTION

The invention relates to novel Evasin polypeptides and their use in inhibiting multiple members of the chemokine network, or detecting chemokine expression and inflammation.

### BACKGROUND OF THE INVENTION

Chemokines are major drivers of inflammation, angiogenesis and fibrosis[1-3], and play key roles in multiple disease processes. These include: atherosclerosis[4], myocardial infarction [5], myocarditis [6], fibrosis and progressive organ failure in the heart, kidney, liver, skin and lungs[3], inflammatory bowel disease[7], chronic obstructive lung disease and asthma[8], multiple sclerosis[9], psoriasis[10], atopic dermatitis[11], age-associated deterioration in cognitive function[12], and cancer growth and metastasis [13, 14]. There are no drugs that effectively target chemokines for treatment of such disorders. Chemokines are secreted peptides that drive inflammation by activating the migration of cells that mediate both innate (e.g. neutrophils, cosinophils, macrophages, monocytes, dendritic cells, mast cells) and adaptive immunity (T cells, B cells) to disease tissues[1, 2, 13]. The 46 chemokines and 19 chemokine G-protein coupled receptors form a complex network that has arisen by gene duplication and divergence[15-17]. Network complexity is driven by the binding of individual chemokines to several receptors and individual receptors to several chemokines [15], and by the expression of several chemokine receptors on individual inflammatory cell types [18].

Network complexity can lead to robustness, i.e. tolerance to error and attack[19]. In the case of the chemokine network, genetic evidence provides support for robustness: combinatorial knockout or depletion of chemokines and/or chemokine receptors are more effective than single knockout or depletion in preventing inflammation in diverse mouse model systems including atherosclerosis[20, 21], asthma[22], contact dermatitis[23], response to influenza[24], experimental autoimmune encephalomyelitis[25] and pulmonary eosinophilia[26]. The idea is also supported by pharmacological evidence: drugs targeting single chemokine network nodes have typically failed in clinical trials of immuno-inflammatory disease [18, 27]. Thus targeting multiple pro-inflammatory chemokine signalling pathways simultaneously may be therapeutically desirable in chemokine-driven inflammation, and may be more effective than targeting a single chemokine or its receptor.

Ticks are efficient bloodsucking parasites that have evolved over ˜250 million years[28] to parasitize a wide host range including amphibians, reptiles, birds, and mammals. Ticks can suck blood for several weeks without eliciting inflammation [29, 30]. There is no acquired resistance to tick infestation, and repeated infestation suppresses the pro-inflammatory TH1 cytokine profile[31-33]. Transcriptomic analyses of tick salivary glands indicate that they have ˜1500-3000 secreted peptides[34, 35]. These include small (10-20 kD) peptides called Evasins that suppress chemokine-driven inflammation by binding and neutralizing multiple chemokines simultaneously[36-39]. Evasins 1, 4 and 3 from Rhiphicephalus sanguineus have been cloned[39]. Evasin4 binds ˜20 CC class chemokines. It does not bind CXC class chemokines[39]. Evasin1 is related to Evasin4, and binds CCL3, CCL4, CCL3L1 and CCL18 [40, 41]. Evasins 1 and 4 target the CCL3 chemokine N-terminus using different pharmacophores and interfere with receptor binding[41, 42]. Evasin3 is unrelated to Evasin1 and 4, and binds CXCL8 and CXCL1 but not CC chemokines[39].

Therapeutic administration of Evasin3 inhibits inflammation in animal models of myocardial ischemia reperfusion injury[43], and plaque vulnerability[44]. Evasin4 improves post-infarction myocardial injury and survival in mice[45]. The Evasins are also effective in mouse models of pancreatitis[46], joint inflammation[39], lung inflammation and fibrosis[47], psoriasis[39], and graft-versus-host disease[48]. The ability to target many chemokines simultaneously is likely the basis for Evasin efficacy in inflammation, and is an important biological property not possessed by alternative anti-chemokine technology including neutralising antibodies[49], nanobodies[49], and dominant negative chemokines[50, 51]. Evasins are substantially smaller (MW=10-20 kD) than viral anti-chemokines, single-chain variable fragment antibodies, or monoclonal antibodies, and are thus likely to exhibit better tissue penetration[52]. Evasins administered subcutaneously have systemic anti-chemokine effects[47] and do not appear to be significantly immunogenic in mice[47]. This makes it likely that they have the potential, like other naturally occurring peptides such as hirudin or exenatide to be translated for clinical therapy[53].

### SUMMARY OF THE INVENTION

The inventors have isolated 31 novel chemokine-binding Evasins peptides of 352 putative Evasins predicted by sequence homology to Evasins 1, 3 and 4 from the publicly available salivary gland transcriptomes of 8 different tick species (Ixodes ricinus, Rhipicephalus sanguineus, Amblyomma maculatum, Amblyomma parvum, Amblyomma triste, Amblyomma americanum, Amblyomma cajennense, Rhipicephalus pulchellus). Novel chemokine-binding Evasins were isolated from a library using yeast surface display technology [54] to express putative Evasins on yeast cell surface and isolating yeast cells binding a specific biotinylated human chemokines and streptavidin-Alex647 using fluorescent-activated cell sorting (FACS) (Table 2 column C). Isolated Evasin expressing yeast clones were reconfirmed to bind indicated chemokines by yeast surface display and flow cytometry (Table 2 column D, FIG. 1). Additional characterisation of human chemokine binding was carried out using biolayer interferometry (FIG. 3) and further evaluation of chemokine neutralisation activity was also performed (FIG. 4).

The chemokine-binding Evasins of the invention have <60% identity to published Evasins 1, 3 and 4, bind different chemokine combinations compared to published Evasins 1, 3 and 4, and are useful for combinatorial targeting of chemokines. The inventor's analysis as shown in FIG. 3 illustrates that the chemokine-binding Evasins of the invention are grouped into distinct functional classes from previously described Evasins, based on distinct chemokine-binding activities. Class I Evasins, represented by the Evasins of SEQ ID NOs 1-3, 6-10, 15-16, 20-23 and 29-30 bind CC-class chemokines CCL2, CCL13 or CCL20 in addition to other chemokines. This binding activity is not reported for any previously described Evasins to the inventor's knowledge, and provides unique binding profiles which may be tailored to desired targeting of human chemokines (also avoiding targeting of certain other chemokines where desired), including in human disease, as shown in Tables 4 and 5. FIG. 5 illustrates the varying chemokine expression shown in various human diseases, and thus the utility of novel classes of Evasins displaying novel binding activities. Class I Evasins are of particular utility for targeting diseases where one or more of CCL2, CCL13 and/or CCL20 are expressed, as discussed below.

Class II Evasins, represented by the Evasins of SEQ ID NOs 5, 19, 24-26, 28 and 31 bind CXC-chemokines 3, 10 or 12 in addition to other chemokines and also provide a binding profile not reported for previously described Evasins, with corresponding implications for novel specificity of targeting of human chemokines, including in human disease. Class II Evasins are of particular utility for targeting diseases where one or more of CXCL3, CXCL10 and/or CXCL12 are expressed, as discussed below.

Class III Evasins, represented by the Evasins of SEQ ID NOs 4, 11-14, 17-18 and 27 provide further novel chemokine-binding agents.

The invention therefore provides a polypeptide comprising (a) all or part of an amino acid sequence shown in any one of SEQ ID NOs: 1 to 31 or (b) all or part of an amino acid sequence having at least 70% homology or amino acid identity to a sequence of (a) over its entire length.

The invention also provides:
a fusion polypeptide comprising a polypeptide of the invention linked to a second peptide or polypeptide;
a combination of two or more polypeptides of the invention;
a polynucleotide which encodes a polypeptide of the invention or a combination of the invention;
a combination of two or more polynucleotides each of which encodes a polypeptide of the invention;
a vector which comprises a polynucleotide of the invention or a combination of the invention;
a host cell which comprises a polynucleotide of the invention, a combination of the invention or a vector of the invention;
a pharmaceutical composition comprising (a) a polypeptide of the invention, a combination of the invention, a polynucleotide of the invention, a vector of the invention or a host cell of the invention and (b) a pharmaceutically acceptable carrier or diluent;
a method of producing a polypeptide of the invention or a combination of the invention comprising, culturing a host cell of the invention under conditions which produce the polypeptide or the combination;
a method of inhibiting the signalling of one or more chemokines in an in vitro culture, the method comprising contacting the culture with a polypeptide of the invention, a combination of the invention, a polynucleotide of the invention, a vector of the invention or a host cell of the invention;
a method of inhibiting the signalling of one or more chemokines in a subject, the method comprising administering to the subject a polypeptide of the invention, a combination of the invention, a polynucleotide of the invention, a vector of the invention or a host cell of the invention;
a method of treating or preventing in a subject one or more diseases associated with one or more chemokines, the method comprising administering to the subject a polypeptide of the invention, a combination of the invention, a polynucleotide of the invention, a vector of the invention or a host cell of the invention.
a polypeptide of the invention, a combination of the invention, a polynucleotide of the invention, a vector of the invention or a host cell of the invention for use in a method of inhibiting the signalling of one or more chemokines in a subject;
a polypeptide of the invention, a combination of the invention, a polynucleotide of the invention, a vector of the invention or a host cell of the invention for use in a method of treating in a subject one or more diseases associated with one or more chemokines;
an antibody or a fragment thereof which specifically binds a polypeptide of the invention;
a method of detecting one or more chemokines in a tissue, comprising contacting the tissue with a detectably-labelled polypeptide of the invention or a detectably-labelled combination of the invention and detecting the binding of the polypeptide or the combination to one or more chemokines; and
a detectably-labelled polypeptide of the invention or a detectably-labelled combination of the invention for use in a method of detecting one or more chemokines in a tissue.

### DESCRIPTION OF THE FIGURES

FIG. 1: Binding of P993_AMBCA to CXCL8 using yeast surface display. Top: Flow sorting of negative control yeast containing an empty yeast surface display vector using CXCL8+streptavidin AF647. Bottom: Flow sorting of P993_AMBCA expressing yeast using CXCL8+streptavidin AF647. X-axis: fluorescent intensity, y-axis: cell count. Note the significant increase in fluorescent intensity with P993_AMBCA. This assay was used to reconfirm chemokine binding characteristics of novel Evasins described in Table 2

FIG. 2: Inhibition of chemokine-induced cell migration by P546_AMBCA. Migration of U2-OS cells (stably transfected with a CCR5 encoding plasmid) in response to human CCL3, CCL4 and CCL5 chemokines (Peprotech) was analysed in 96-well format using a IncuCyte ZOOM® chemotaxis system over 48 hours, and progress curves recorded. The area under the progress curve (AUC) was calculated using GraphPad Prism, and plotted on the y-axis. Results of 3 experiments are shown as mean+SEM. Statistical significance was calculated using the t-test, * indicates p<0.05, ** p<0.05.

FIG. 3A-C: Binding characteristics of novel Evasins. All members of the human chemokine family [18)] are listed in column 1. Binding to human chemokines was determined for 14 novel evasins using biolayer interferometry (BLI) [126] with calculated Kd shown as Molar (Moles/Litre). Data is shown in FIGS. 3A and B. Binding data for 17 other novel evasins was assayed using yeast surface display [124], with positive binding results shown as “YES” in FIG. 3C. For biolayer interferometry, His-tagged purified evasin was bound to a Ni-NINTA sensor on an OctetRed® 384 system, and then binding to each chemokine listed (with the exception of CCL25, CCL26, CXCL16, CXCL17, CXCL4L1, XCL2) was assayed in a cross-binding screen at a chemokine concentration of 300 nM. For those chemokines showing binding to a evasin in the cross-binding screen, binding assays were repeated using different doses of chemokine. Association, equilibrium and dissociation data were analysed using Octet software to create corresponding fitted curves, and used to calculate Kd. For yeast surface display, (YSD) background fluorescence was controlled for by using either an empty vector or by omitting the chemokine (i.e. using streptavidin-Alexa647 alone). An arbitrary threshold of >3 fold over background mean fluorescence intensity was chosen to describe confirmed re-tests. Where binding was detected data are indicated as “YES”. For biolayer interferometry (BLI) data, empty cells in FIG. 3A-C represent chemokines where either binding assays were not done (CCL25, CCL26, CXCL16, CXCL17, CXCL4L1, XCL2); or where no binding was confirmed by biolayer interferometry. For yeast surface display data, empty cells represent chemokines that were not tested.

Binding data published in relation to previously described Evasins (Evasins 1, 4 and 3) is also shown for comparison in each of FIGS. 3A-C (see data for “Previously described Evasins”, with the relevant data obtained from publications: [39-41 and 125].

FIG. 4: Neutralisation of human chemokines by novel Evasins. Neutralising activity was determined using a quantitative THP1 cell migration assay in a 96-well Boyden chamber with chemokine in the bottom chamber. Cells migrating through to the bottom chamber at 4 hours were counted using flow cytometry in a 96 well plate format.

IC50 for neutralisation was determined at the chemokine EC80 dose as determined using a range of evasin concentrations. Data was analysed using GraphPad Prism to determine IC50, which is shown as Molar (Moles/Litre). Empty cells represent experiments not done.

FIG. 5: Expression patterns of chemokines in disease states. Data for positive expression (shown as filled squares) are taken from the published literature. Where a novel evasin binding pattern maps on to a given chemokine expression pattern in disease, this provides indications for therapeutic use of the novel evasin. Literature references are: Giant cell myocarditis [116, 117]; Chagas myocarditis [113]; heart allograft rejection [118]; myocarditis [115,118-120,127]; myocardial infarction [128]; ANCA vasculitis [129-133]; giant cell arteritis [129, 134-136]; Takayasu disease [137, 138]; atherosclerosis [58-72]; stroke [139]; multiple sclerosis [93-95]; Alzheimer disease [112]; autoimmune hepatitis [140]; primary biliary cirrhosis [141-147]; primary sclerosing cholangitis [144,148]; liver fibrosis [140, 149]; non-alcoholic steatohepatitis (NASH) [45, 130]; paracetamol liver injury [151]; alcohol liver injury [152]; idiopathic pulmonary fibrosis [153-163]; kidney fibrosis [149]; inflammatory bowel disease [74-84]; rheumatoid arthritis [73, 164]; psoriasis [108-111]; atopic dermatitis [86-87, 165-166]; breast cancer [96-102]; colorectal cancer [103-107].

### DESCRIPTION OF THE SEQUENCE LISTING

SEQ ID NOs: 1 to 31 are shown in Table 1 below and in the ST.25 format electronic sequence listing which forms part of the application.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms' “a” “an”, and “the” ˜ include plural referents unless the content clearly dictates otherwise. Thus, for example, reference to “a polypeptide” includes two or more such polypeptides, or reference to “a polynucleotide” includes two or more such polynucleotides and the like.

All publications, patents and patent applications cited herein, whether supra or infra, are hereby incorporated by reference in their entirety.

### Evasins of the Invention

TABLE 1
Novel Evasins of the invention. Tick peptide sequences
isolated in yeast surface display fluorescent - activated
cell sorting (FACS) screens using a labelled chemokine.
Identity with Evasin 1, 4 or 3 was calculated using BLAST.

| SEQ | | | Identity with |
|---|---|---|---|
| ID | | | Evasin 11, 4 or |
| NO: | Peptide Name | Peptide Sequence | 3 using BLAST |
| 1 | P467_RHIPU | AEKSLDSDSSGEDYELWTQGCPFLVAENR | EVA1(53.09%); |
| | | TGFGTTVSCQHNCNGAIEKVPEGEPCYTIG | EVA4(35.14%); |
| | | EDGLGRNIKLNLPYNCSLGECSGGVCVPNG | EVA3(35.48%) |
| | | RSDVCFKRTWEENNKAMA | |
| 2 | P546_AMBCA | ENTQQEEEDYDYGTDTCPFPVLANKTNKA | EVA1(41.30%); |
| | | KFVGCHQKCNGGDQKLTDGTACYVVERK | EVA4(36.36%) |
| | | VWDRMTPMLWYSCPLGECKNGVCEDLRK | |
| | | KEECRKGNGEEK | |
| 3 | P672_RHIPU | VCEVSEQEGVGEDNATEDEDYEDFFKPVT | EVA4(36.19%); |
| | | CYFANSTVGPLRPPNCTVVCTNNTAWWN | EVA1(29.21%) |
| | | DTKSDGGHCYSEYRPEKRTHSREIYNCTIG | |
| | | VCGNGTCIANHTYADCW | |
| 4 | P698_RHISA | NEDDSSDYYDASPMNCSSMSVNSTMGWL | EVA1(30.68%); |
| | | SMNCTMSCNGTTFPLSNSTHCFHSYTNLTV | EVA4(35.48%) |
| | | QSRMETMTYNCSVGTCSNGTCVENGTTTT | |
| | | CW | |
| 5 | P943_IXORI | RSKQPTASQSSKNSIKAEFCDTNCTQGTNG | EVA3(50.00%) |
| | | AWSGCSEGCFCVHVGNNTKGRCMKLSSD | |
| | | YDYTTQ | |
| 6 | P974_AMBCA | ENTQQEEQDYDYGTDTCPFPVLANKTNKA | EVA1(41.30%); |
| | | KFVGCHQKCNGGDQKLTDGTACYVVERK | EVA4(36.36%) |
| | | VWDRMTPMLWYECPLGECKNGVCEDLRK | |
| | | KEDCRKGNGEEK | |
| 7 | P983_AMBCA | EDTGTEDDFDYGNTGCPFPVLGNYKSNMT | EVA1(39.77%); |
| | | KPVGCKNKCGSGYEVLNDTTPCYVIDQKV | EVA4(25.00%) |
| | | FNNMVPLRQYSKCPLGFCENGECKPNDQA | |
| | | EDCYKGREEQK | |
| 8 | P985_AMBPA | DEESEELGASTDVDYEELDANCTCPAPALT | EVA1(28.57%); |
| | | STRNNKHYPLGCIYNCSSYNCTIPDGTPCY | EVA4(29.91%) |
| | | VLTLGEVKEHLQIGSTVPNCTCGLCRNGTC | |
| | | VSNGTVEECFAVEEIEET | |
| 9 | P991_AMBCA | ENGEGTTQPDYDNSTDYYNYEDFKCTCPA | EVA1(30.00%); |
| | | PHLNNTNGTVMKPIGCYYTCNVTRCTAPD | EVA4(25.53%) |
| | | TYPCYNLTEHQAKNLTTSPTTLCAVGNCD | |
| | | HGICVPNGTKELCFKAPNLEE | |
| 10 | P993_AMBPA | HRHYDPNEPCLIAGLKTPRDALPAGCRYDC | EVA1(33.85%); |
| | | MIKKNQKLRDGLLCLDVPEKVVKRMVNY | EVA4(36.36%) |
| | | LNYSCPLGTCRKGICKRKHRNVRCQKYPV | |
| | | FYMSPPK | |
| 11 | P1005_AMBMA | EKDTPKNIPGCGDTGTTAAPPADNPKHFAV | EVA4(26.60%); |
| | | YTDKHGCTIKVIGTWMTKEDHSRLPVSLR | EVA1(27.12%); |
| | | RVRASSGRVMLPASCQKICNDTVKNFPEGT | EVA3(39.13%) |
| | | PCRLVTGDPIKGKNHIKDGCIRGYCSSGVC | |
| | | VSDKRNISCYVPPN | |
| 12 | P1006_AMBCA | DTIGGIPGCGDPATTSAPEDQPKHYAVRTD | EVA1(38.33%); |
| | | KNGCKVMVIGTWMTKEDHNLLPPYISKER | EVA4(20.22%) |
| | | APSGKVQLPASCKKNCHGKLKNLPNGTPC | |
| | | REVFGDLRRRRKHIKDGCKVGKCQNGLCV | |
| | | SEERIISCYLPPNITDPRPTHGPLAE | |
| 13 | P1011_AMBTR | EKDTPKNIPGCEDAGTTAAPPADNPKHYA | EVA4(26.51%); |
| | | VYTDKNGCTFKVIGTWMTKEDHRRLPVSL | EVA1(23.73%); |
| | | RRVRASSGRVKLPASCQKICKHSVKKFPEG | EVA3(34.62%) |
| | | TPCRLVTRDPIKGKNHIKNGCIRGYCSSGV | |
| | | CVSDNRSISCYVLPNNTELTSPSGSFAE | |
| 14 | P1014_AMBCA | DIIGGIPGCGDPTTTSAPEVPPKHFAVRTDK | EVA1(30.23%); |
| | | DGCTLMIIGTWMTTEDHNRLPPDIGEKRAP | EVA4(36.36%); |
| | | YGRVQLPASCKKNCHGKVKNLPNGTPCRE | EVA3(33.33%) |
| | | VFGDPRRGRKHIKNGCTVGKCQSGLCVTD | |
| | | KRIISCYIPPNITDPIPTHGPWAE | |
| 15 | P1015_AMBCA | EEAPGPAPGCGEPEPTPPKPRRHGIVTNVNS | EVA4(32.39%); |
| | | CNSTILVWNGKEFPALCKVRCPHKSYRVS | EVA1(25.76%) |
| | | DFEPCLKFTNRRLQERKDETPYKCKLGFC | |
| | | RHGTCITSEHSRKVPCKVPADRLDPSE | |
| 16 | P1030_AMBPA | GPPSIPGNESIPGCGDAGTTTAPEDNPKHYG | EVA1(24.59%); |
| | | TLTDKKGCTLPIIGTWMTAVDHQHLQGTR | EVA3(37.93%) |
| | | GERRGPTGKVNLPASCRKNCHGRQEILRD | |
| | | GIPCRKVVGNPKGSKKHLKSGCLRGKCLA | |
| | | GQCVNDGRRISCYVPRNITDTEPTPGLLAE | |
| 17 | P1063_AMBTR | QSEVGKNVPGCGDTETLEAPPQEKPPYYEY | EVA1(30.61%); |
| | | KDEEGCTQKVLESWFQAGERSTGRGQKKR | EVA3(43.48%); |
| | | GHGRRPSRVLRTVDCRKNCTVGITALPDG | EVA4(26.42%) |
| | | HLCLVPRGDPFTRGGAIKYGCYLGDCASG | |
| | | HCQHRYETVSCRLPAPDTTAKPYYVTPEK | |
| 18 | P1146_IXORI | GPDTKGDEESDENELFTVEYCGTNCTQLEN | None detected |
| | | GSWTPCSGNNGNCRCFEIESDKTVGLCLST | |
| | | EYTDFSEYPDPNSSEIIAAAPLPRERLIQ | |
| 19 | P1156_IXORI | ADDDNELFTVQYCGMNCTKDEGGTWTGC | EVA3(30.23%) |
| | | TGKKEGCKCYHESGKNYGLCLSTEYTDFS | |
| | | QYGNPSDSEIEAAKPKRSDTLSH | |
| 20 | P1180_AMBTR | EEPKDGYDYTEGCPFVVLGNGTHAKPAGC | EVA1(40.74%); |
| | | SHLCNGAPETLDDNMECYNVTEEVAKRM | EVA4(28.74%) |
| | | TPDIPYTCWLGWCSKGECKRDNRTEVCYR | |
| | | GSERE | |
| 21 | P1181_AMBMA | EEREDDNDYGGGCPFVVLGNGTHAKPAGC | EVA1(45.05%); |
| | | SHLCNGAPETLDNIECYNVTEEVAKRMTP | EVA4(24.73%) |
| | | DIPYTCWLGWCSKGECKRDNRTEVCYRGS | |
| | | ERE | |
| 22 | P1182_AMBMA | EPKDDNDYGGGCPFVVLGNGTHAKPAGCS | EVA1(44.19%); |
| | | HLCNGAPETLDNIECYNVTEEVAKRMTPGI | EVA4(24.18%) |
| | | PYACWLGWCNKGECKRGNRTEVCYRGSE | |
| | | EE | |
| 23 | P1183_AMBTR | EAPKDDFEYDGGCPFVVLDNGTHVKPAGC | EVA1(43.02%); |
| | | SHLCNGAPETLDNIECYNVTEEVAKRMTP | EVA4(25.00%) |
| | | GIPYACWLGWCSKGECKRDNRTEVCYRGS | |
| | | EEE | |
| 24 | P1209_AMBPA | KTDTKNAAGELPPKVAIPGCEDPATTKAPL | EVA1(35.59%); |
| | | PDDPRYYGVTIDKDGCQRKVLGSSQRQQR | EVA4(22.22%) |
| | | QVQNGRKPGRKGRGRRPVFVDLKLTVDC | |
| | | KRKCNGTYSQLPDGEPCLVCDGEPYGRHR | |
| | | TIKGGCYQGNCSSGQCHRGERKVNCYIPK | |
| | | NITNNVLNSVNLAE | |
| 25 | P1215_AMBPA | KTDTKNAAGELPPKVVIPGCEDPATTKAPL | EVA1(33.90%); |
| | | PDEPPYYGITIDKDGCQRKVLGSSQRQQRK | EVA4(20.83%) |
| | | VQNGRKLDGKKRGRRPVFVDLELTVDCKR | |
| | | KCNGTYSQLPDGENCLVSDGYPYGRWGTI | |
| | | KGGCYQGNCSSGQCHRGEKKVNCYLPKNI | |
| | | TNDVPKSLNLAE | |
| 26 | P1219_IXORI | ASLAKETEDTTLPARALVDSPDSDNCSSPQ | EVA4(32.00%) |
| | | LPYFDELTYMPLGFLAVNCTKTCPVGKNG | |
| | | TVVNGNKCIVTWSILDVSTITVLVGSCKNG | |
| | | YCISDGSSECRNITLAGEDSQEEEEEAEEDE | |
| | | EEDDGDEEEDEEEDEEENDDD | |
| 27 | P1220_IXORI | GSTPSAMKTEDILKVLGSTSSLENHTDSSH | EVA3(30.43%) |
| | | CRYQELLDITKNIENAGFLAINCQRSCPNG | |
| | | KQTMVEGYGCIFKIKHATKRGKVKVKEGS | |
| | | CRKGACVRGSTRPPWRLLVLLGESKEEEFL | |
| 28 | P1224_IXORI | SDLCKMEAESSPFKLPQSSLLDAPDEEGCK | EVA1(25.33%); |
| | | YQLLFVEAEGPLVVNCTKDCPNGKIRTVVE | EVA3(33.33%) |
| | | GELCIAMVKTSSSGEATGLVGSCKRGSCVK | |
| | | KDDPCRTFTLSEEGDDDEEDEEEEEDEEED | |
| | | EEEDEEEEEEDEEEED | |
| 29 | P1243_AMBAM | RNHTEDNSTEYYDYEEARCACPARULNNT | EVA1(32.79%); |
| | | NGTVLKLLGCHYFCNGTLCTAPDGYPCYN | EVA4(42.86%) |
| | | LTAQQVRTLTTYPNTSCAVGVCMKGTCVK | |
| | | NGTMEQCFKTP | |
| 30 | P1252_AMBAM | RGGAASVPANASIPGCGDAQTTPAAPEDQP | EVA1(30.00%); |
| | | KHYVVYRDGNGCEVKIIGTWMTTEDYNCL | EVA4(24.24%) |
| | | PDTFRKNRAPHGKVKLPASCKKTCGNAVQ | |
| | | NLKDGTPCRKVFGDLGRRRNLIKNGCLVG | |
| | | ACQSGLCVSGNRTISCYIPPNSTDTRATPGS | |
| | | FAE | |
| 31 | P1283_IXORI | KEPEDTTLPPGALVDSPDSDNCSSPHLPYFD | None detected |
| | | ETTNMWMGFLAVNCTKKCPVGKHVTVVD | |
| | | GNKCIGTWSFLDELTITVLVGSCKDGFCET | |
| | | DGSSECRNITLAEEDSQEEEGAAAEEEDEE | |
| | | DEEDEEEEEAEEEREDDHDDA | |

Abbreviations:
IXORI—Ixodes ricinus,
RHISA—Rhipicephalus sanguineus,
AMBMA—Amblyomma maculatum,
AMBPA—Amblyomma parvum,
AMBTR—Amblyomma triste,
AMBAM—Amblyomma americanum,
AMBCA—Amblyomma cajennense,
RHIPU—Rhipicephalus pulchellus.

TABLE 2
Chemokines inhibiting by the novel Evasins. Mapping of isolated tick peptide sequences to chemokine
FACS screen and to FACS re-testing data using yeast surface display. For re-testing, background
fluorescence was controlled for by using either an empty vector or by omitting the chemokine
(i.e. using streptavidin-Alexa647 alone). An arbitrary threshold of >3 fold over background
mean fluorescence intensity was chosen to describe confirmed re-tests. Note that for certain
tick peptides a more exhaustive retesting was performed using a wider panel of chemokines.

| A: | | C: | D: |
|---|---|---|---|
| SEQ | | Chemokine FACS screens | Chemokines reconfirmed to bind to |
| ID | B: | that the peptide was | peptide at >3 fold mean fluorescent |
| NO: | Peptide Name | isolated in | intensity over control |
| 1 | P467_RHIPU | CCL1; CCL2; CCL3; CCL5 | CCL2 |
| 2 | P546_AMBCA | CCL1; CCL3; CCL5; CCL22 | CCL1; CCL2; CCL3; CCL4; CCL5; CCL8; |
| | | | CCL17; CCL18; CCL19; CCL22; CCL25; |
| | | | CX3CL1 |
| 3 | P672_RHIPU | CCL8 | CCL8 |
| | | | CCL8 |
| 4 | P698_RHISA | CCL5 | CCL5; CCL8 |
| 5 | P943_IXORI | CCL5 | CCL5 |
| 6 | P974_AMBCA | CCL1; CCL3; CCL4; CCL8; | CCL8; CCL17 |
| | | CCL17; CCL18; CCL22 | CCL3; CCL4 |
| 7 | P983_AMBCA | CCL2; CCL3; CCL8 | CCL8 |
| | | | CCL2; CCL3; CCL4; CCL8; CCL11 |
| 8 | P985_AMBPA | CCL5 | CCL3; CCL4; CCL5; CCL8; CCL11; |
| | | | CCL17; CXCL10; CXCL11 |
| 9 | P991_AMBCA | CCL2; CCL4; CCL5; CCL8; | CCL5; CCL8; CCL17 |
| | | CCL17; CCL18; CCL19; | CCL2; CCL3; CCL5; CCL8; CCL11; |
| | | CCL22 | CCL17; CCL18; CCL19; CXCL12 |
| 10 | P993_AMBPA | CCL1; CCL11; CCL17; | CCL5; CCL17; CCL20 |
| | | CCL20; CCL25; CXCL8; | CCL20; CXCL8 |
| | | CX3CL1 | |
| 11 | P1005_AMBMA | CCL1; CCL11; CCL25; | CCL1; CCL3; CCL5; CCL11; CCL17; |
| | | CX3CL1 | CCL19; CXCL8 |
| 12 | P1006_AMBCA | CCL1; CCL11; CCL25; | CCL5 |
| | | CXCL8; CX3CL1 | CCL15; CCL19; CCL22; CCL25 |
| 13 | P1011_AMBTR | CCL11; CX3CL1 | CCL5; CCL11; CCL17; CCL18; CCL25 |
| 14 | P1014_AMBCA | CCL1; CCL11; CCL25; | CCL4 |
| | | CX3CL1 | |
| 15 | P1015_AMBCA | CCL20; CXCL12 | CCL20; CXCL12 |
| | | | CCL20 |
| 16 | Pl030_AMBPA | CCL11; CCL17; CX3CL1 | CCL17 |
| | | | CCL3; CCL5; CCL8; CCL11; CCL18; |
| | | | CCL20; CXCL8 |
| 17 | P1063_AMBTR | CCL5 | CCL5 |
| 18 | P1146_IXORI | CXCL8 | CXCL8 |
| 19 | P1156_IXORI | CXCL8 | CXCL8 |
| 20 | P1180_AMBTR | CCL2; CCL3; CCL4; CCL8; | CCL8 |
| | | CCL18 | CCL3; CCL8 |
| 21 | P1181_AMBMA | CCL3; CCL4 | CCL3; CCL4 |
| 22 | P1182_AMBMA | CCL2; CCL3; CCL4; CCL8; | CCL8 |
| | | CCL18 | CCL3; CCL4; CCL8 |
| 23 | P1183_AMBTR | CCL2 | CCL2; CCL11 |
| 24 | P1209_AMBPA | CCL1; CCL5; CCL11; | CXCL12 |
| | | CXCL8; CXCL12 | CCL1; CCL5; CCL8; CCL11; CCL17; |
| | | | CCL18; CCL19; CCL20; CCL25; CXCL8; |
| | | | CXCL10; CXCL12; CX3CL1 |
| 25 | P1215_AMBPA | CCL1; CCL11; CXCL8; | CCL5 |
| | | CX3CL1 | CCL5; CCL11; CCL18; CXCL12 |
| 26 | P1219_IXORI | CCL19; CXCL10 | CXCL10 |
| 27 | P1220_IXORI | CCL25; CX3CL1 | CCL25 |
| 28 | P1224_IXORI | CCL3; CCL19; CXCL10 | CXCL10 |
| 29 | P1243_AMBAM | CCL4; CCL15; CCL17; | CCL17 |
| | | CCL18 | CCL3; CCL4; CCL15; CCL17; CCL19 |
| 30 | P1252_AMBAM | CCL11; CCL17; CCL20 | CCL17; CCL20 |
| 31 | P1283_IXORI | CXCL10 | CXCL10 |

TABLE 3
Binding of novel Evasins to chemokines using microscale
thermophoresis. HEK293T cells (ATCC) were transiently
transfected using Fugene (Promega) following the manufacturers
instructions, and proteins expressed essentially as
described[55]. Proteins were purified from supernatants
using HisMag sepharose excel beads (GE Healthcare) and
then MagStrep beads (IBA) essentially as recommended
by the manufacturer, or using gel filtration. Microscale
thermophoresis [56, 57] was performed using a
Nanotemper NT.115 Monolith instrument following the
manufacturers recommendations, using AlexaFluor 647
labelled chemokines (Almac). Data was fitted using NT
Affinity software using thermophoresis and T-jump parameters
and the kD was estimated.

| SEQ ID NO: | Peptide name | Chemokine | Kd_nM |
|---|---|---|---|
| 23 | P1183_AMBTR | CCL2_HUMAN | 77 |
| 29 | P1243_AMBAM | CCL15_HUMAN | 4 |
| 29 | P1243_AMBAM | CCL17_HUMAN | Not |
| | | | detected |
| 29 | P1243_AMBAM | CCL18_HUMAN | Not |
| | | | detected |
| 4 | P698_RHISA | CCL5_HUMAN | Not |
| | | | detected |
| 25 | P1215_AMBPA | CCL5_HUMAN | 73 |
| 8 | P985_AMBPA | CCL5_HUMAN | 48 |
| 1 | P467_RHIPU | CCL5_HUMAN | 48 |
| 2 | P546_AMBCA | CCL1_HUMAN | 149 |
| 2 | P546_AMBCA | CCL2_HUMAN | 682 |
| 2 | P546_AMBCA | CCL3_HUMAN | 171 |
| 2 | P546_AMBCA | CCL5_HUMAN | 128 |
| 2 | P546_AMBCA | CCL8_HUMAN | 80 |
| 2 | P546_AMBCA | CCL11_HUMAN | 500 |
| 2 | P546_AMBCA | CCL18_HUMAN | 86 |
| 2 | P546_AMBCA | CCL19_HUMAN | 367 |
| 2 | P546_AMBCA | CCL20_HUMAN | 232 |
| 2 | P546_AMBCA | CCL22_HUMAN | 175 |
| 2 | P546_AMBCA | CCL25_HUMAN | 1218 |
| 2 | P546_AMBCA | CX3CL1_HUMAN | 140 |
| 2 | P546_AMBCA | CXCL8_HUMAN | 56 |
| 2 | P546_AMBCA | CXCL10_HUMAN | 104 |
| 2 | P546_AMBCA | CXCL11_HUMAN | Not |
| | | | detected |
| 2 | P546_AMBCA | CXCL12_HUMAN | 71 |

TABLE 4
A partial list of potential disease applications of novel Evasins. Table based on
published chemokine expression in disease states (see references, Atherosclerosis
[58-72]. Rheumatoid arthritis [73], Inflammatory bowel disease[73-82]
[77, 83-85], Atopic Dermatitis[86-89] [90-92], Liver fibrosis[3],
Lung fibrosis[3], Kidney fibrosis[3], Skin fibrosis[3], Multiple sclerosis
[93-95], Breast cancer[96, 97], [97-102], Colorectal cancer [103-107],
Psoriasis[87, 108-111], Alzheimer[112], Myocarditis[113-120]) and binding
of novel evasin to chemokine in yeast surface display experiment.

| 1: SEQ | 2: Peptide | |
|---|---|---|
| ID NO: | Name | 3: Disease(s) |
| 1 | P467_RHIPU | Atherosclerosis; Rheumatoid arthritis; Inflammatory bowel |
| | | disease; Liver fibrosis; Lung fibrosis; Kidney fibrosis; Skin |
| | | fibrosis; Multiple sclerosis; Breast cancer; Alzheimer; Myocarditis |
| 2 | P546_AMBCA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast |
| | | cancer; Psoriasis; Alzheimer; Myocarditis |
| 3 | P672_RHIPU | Rheumatoid arthritis |
| 4 | P698_RHISA | Atherosclerosis; Rheumatoid arthritis; Atopic Dermatitis; Liver |
| | | fibrosis; Lung fibrosis; Kidney fibrosis; Skin fibrosis; Multiple |
| | | sclerosis; Breast cancer; Alzheimer; Myocarditis |
| 5 | P943_IXORI | Atherosclerosis; Rheumatoid arthritis; Atopic Dermatitis; Liver |
| | | fibrosis; Lung fibrosis; Kidney fibrosis; Skin fibrosis; Multiple |
| | | sclerosis; Breast cancer; Alzheimer; Myocarditis |
| 6 | P974_AMBCA | Atherosclerosis; Rheumatoid arthritis; Inflammatory bowel |
| | | disease; Atopic Dermatitis; Liver fibrosis; Lung fibrosis; Skin |
| | | fibrosis; Multiple sclerosis; Breast |
| | | cancer; Psoriasis; Alzheimer; Myocarditis |
| 7 | P983_AMBCA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast |
| | | cancer; Psoriasis; Alzheimer; Myocarditis |
| 8 | P985_AMBPA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast |
| | | cancer; Psoriasis; Alzheimer; Myocarditis |
| 9 | P991_AMBCA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast cancer; Colorectal |
| | | cancer; Alzheimer; Myocarditis |
| 10 | P993_AMBPA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast cancer; Colorectal |
| | | cancer; Psoriasis; Alzheimer; Myocarditis |
| 11 | P1005_AMBMA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast |
| | | cancer; Psoriasis; Alzheimer; Myocarditis |
| 12 | P1006_AMBCA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast |
| | | cancer; Alzheimer; Myocarditis |
| 13 | P1011_AMBTR | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast |
| | | cancer; Alzheimer; Myocarditis |
| 14 | P1014_AMBCA | Atherosclerosis; Inflammatory bowel disease; Multiple |
| | | sclerosis; Breast cancer; Psoriasis |
| 15 | P1015_AMBCA | Atherosclerosis; Rheumatoid arthritis; Inflammatory bowel |
| | | disease; Lung fibrosis; Multiple sclerosis; Breast cancer; Colorectal |
| | | cancer; Psoriasis; Myocarditis |
| 16 | P1030_AMBPA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast cancer; Colorectal |
| | | cancer; Psoriasis; Alzheimer; Myocarditis |
| 17 | P1063_AMBTR | Atherosclerosis; Rheumatoid arthritis; Atopic Dermatitis; Liver |
| | | fibrosis; Lung fibrosis; Kidney fibrosis; Skin fibrosis; Multiple |
| | | sclerosis; Breast cancer; Alzheimer; Myocarditis |
| 18 | P1146_IXORI | Atherosclerosis; Rheumatoid arthritis; Inflammatory bowel |
| | | disease; Skin fibrosis; Breast cancer; Psoriasis; Alzheimer |
| 19 | P1156_IXORI | Atherosclerosis; Rheumatoid arthritis; Inflammatory bowel |
| | | disease; Skin fibrosis; Breast cancer; Psoriasis; Alzheimer |
| 20 | P1180_AMBTR | Atherosclerosis; Rheumatoid arthritis; Inflammatory bowel |
| | | disease; Liver fibrosis; Lung fibrosis; Skin fibrosis; Multiple |
| | | sclerosis; Alzheimer |
| 21 | P1181_AMBMA | Atherosclerosis; Rheumatoid arthritis; Inflammatory bowel |
| | | disease; Liver fibrosis; Lung fibrosis; Skin fibrosis; Multiple |
| | | sclerosis; Breast cancer; Psoriasis; Alzheimer |
| 22 | P1182_AMBMA | Atherosclerosis; Rheumatoid arthritis; Inflammatory bowel |
| | | disease; Liver fibrosis; Lung fibrosis; Skin fibrosis; Multiple |
| | | sclerosis; Breast cancer; Psoriasis; Alzheimer |
| 23 | P1183_AMBTR | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast |
| | | cancer; Alzheimer; Myocarditis |
| 24 | P1209_AMBPA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast cancer; Colorectal |
| | | cancer; Psoriasis; Alzheimer; Myocarditis |
| 25 | P1215_AMBPA | Atherosclerosis; Rheumatoid arthritis; Inflammatory bowel |
| | | disease; Atopic Dermatitis; Liver fibrosis; Lung fibrosis; Kidney |
| | | fibrosis; Skin fibrosis; Multiple sclerosis; Breast cancer; Colorectal |
| | | cancer; Alzheimer; Myocarditis |
| 26 | P1219_IXORI | Atherosclerosis; Rheumatoid arthritis; Inflammatory bowel |
| | | disease; Liver fibrosis; Multiple |
| | | sclerosis; Psoriasis; Alzheimer; Myocarditis |
| 27 | P1220_IXORI | Atherosclerosis; Inflammatory bowel disease |
| 28 | P1224_IXORI | Atherosclerosis; Rheumatoid arthritis; Inflammatory bowel |
| | | disease; Liver fibrosis; Multiple |
| | | sclerosis; Psoriasis; Alzheimer; Myocarditis |
| 29 | P1243_AMBAM | Atherosclerosis; Rheumatoid arthritis; Inflammatory bowel |
| | | disease; Atopic Dermatitis; Liver fibrosis; Lung fibrosis; Skin |
| | | fibrosis; Multiple sclerosis; Breast |
| | | cancer; Psoriasis; Alzheimer; Myocarditis |
| 30 | P1252_AMBAM | Atherosclerosis; Rheumatoid arthritis; Inflammatory bowel |
| | | disease; Atopic Dermatitis; Skin fibrosis; Breast cancer; Colorectal |
| | | cancer; Psoriasis; Myocarditis |
| 31 | P1283_IXORI | Atherosclerosis; Rheumatoid arthritis; Inflammatory bowel |
| | | disease; Liver fibrosis; Multiple |
| | | sclerosis; Psoriasis; Alzheimer; Myocarditis |

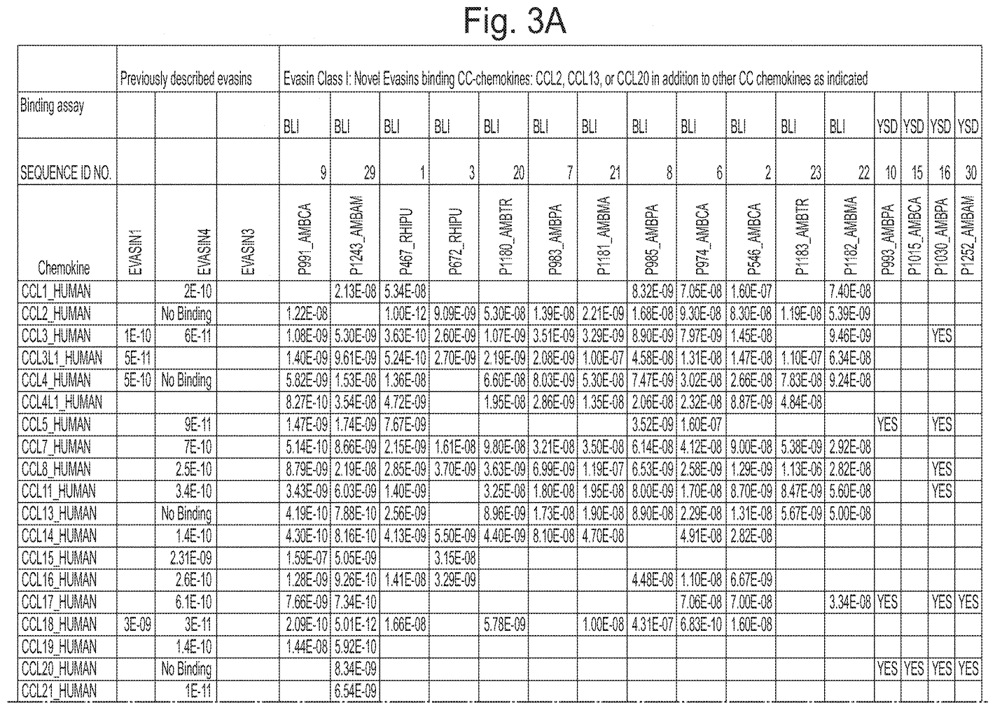

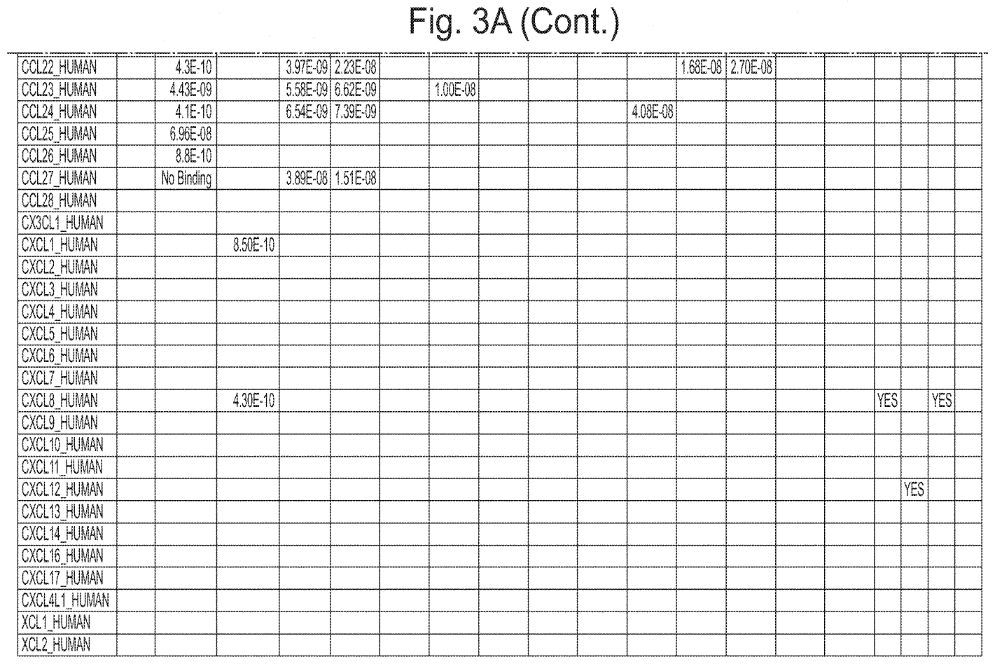

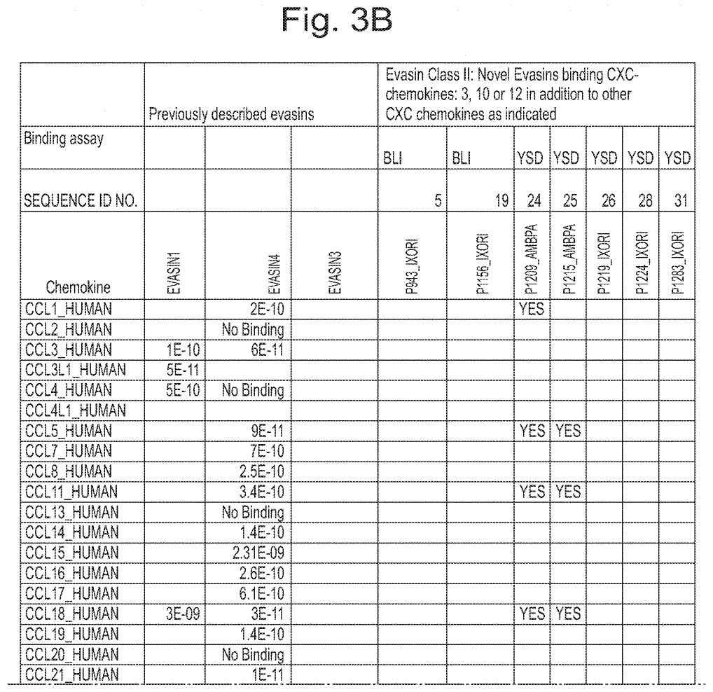

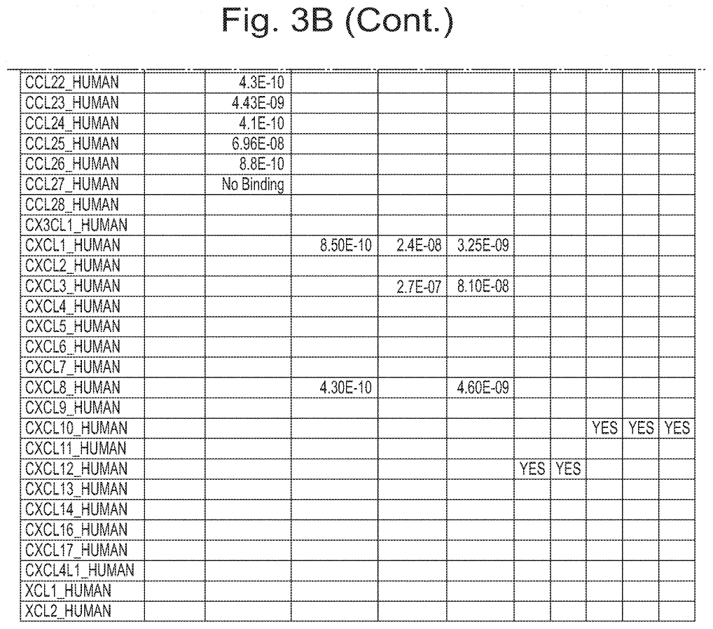

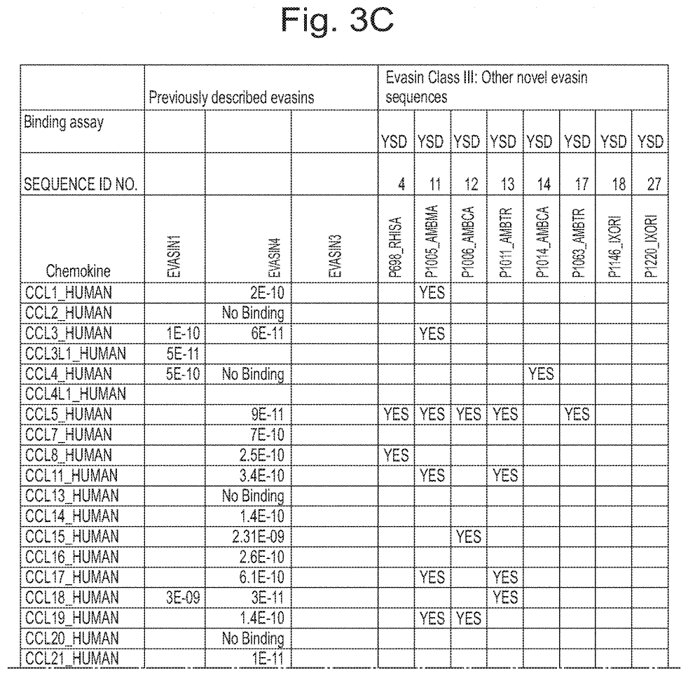

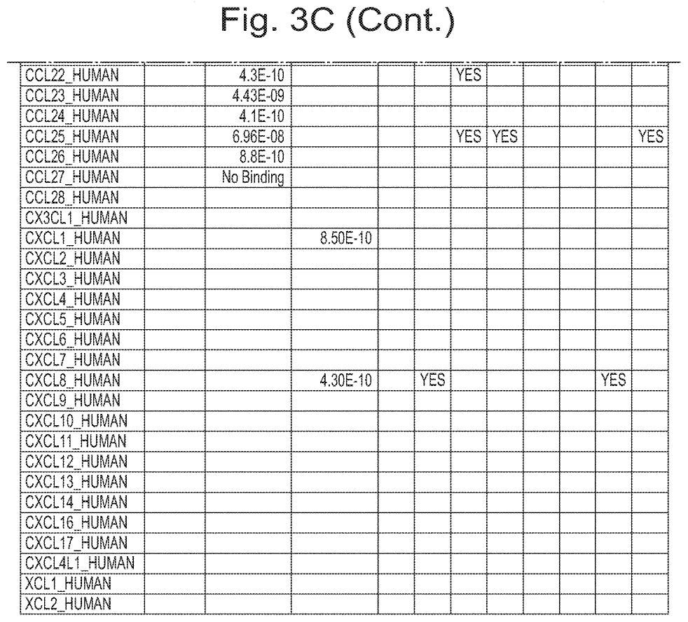

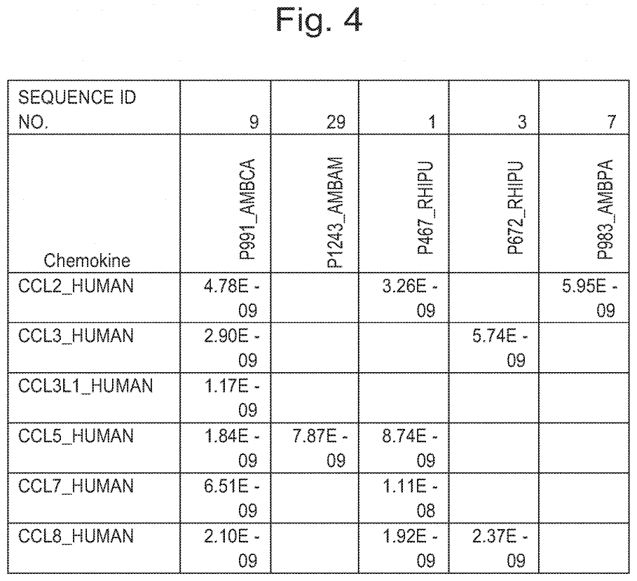

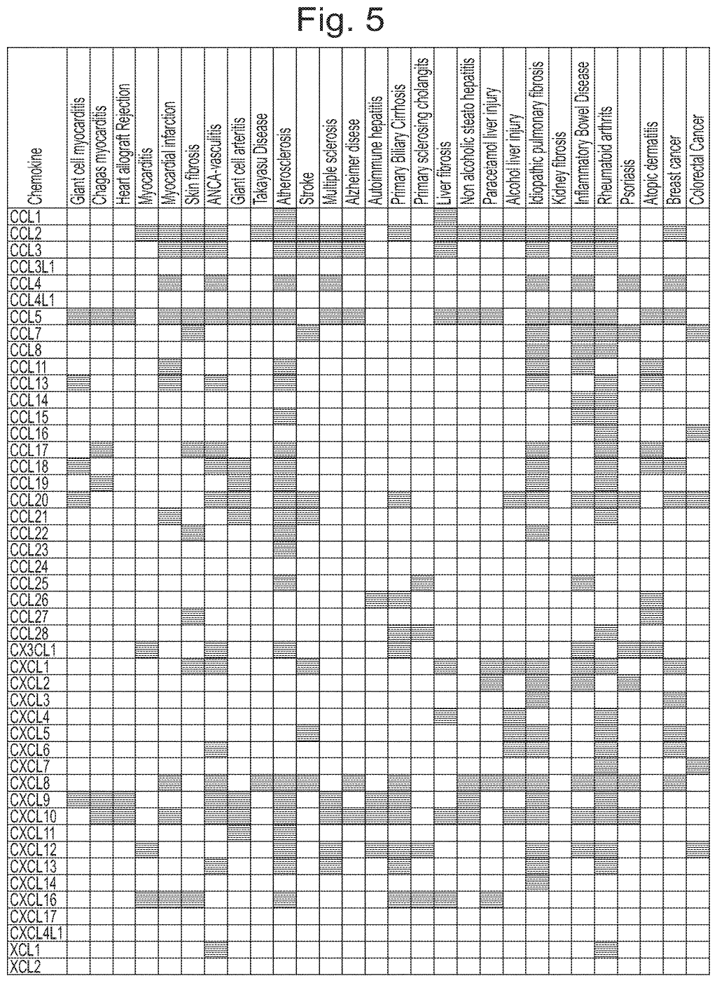

TABLE 5
Additional list of potential disease applications of novel Evasins. Table based
on binding and inhibition data shown in FIGS. 3 and 4 and on published chemokine
expression in disease states as shown in FIG. 5. References for chemokine expression
in disease are as discussed above in relation to FIG. 5.

| _1:_SEQ_ID_NO: | _2:_Peptide_Name | Diseases |
|---|---|---|
| 1 | P467_RHIPU | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer disease; Chagas |
| | | Myocarditis; Myocarditis; Giant Cell |
| | | Myocarditis; Heart allograft |
| | | rejection; Primary biliary cirrhosis; ANCA |
| | | Vasculitis; Giant cell arteritis; Non |
| | | alcoholic steatohepatitis; Takayasu |
| | | disease; Myocardial Infarction; Alcohol |
| | | liver injury; Paracetamol liver injury; Stroke |
| 2 | P546_AMBCA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer disease; Chagas |
| | | Myocarditis; Myocarditis; Giant Cell |
| | | Myocarditis; Heart allograft |
| | | rejection; Primary biliary cirrhosis; Primary |
| | | sclerosing cholangitis; ANCA |
| | | Vasculitis; Giant cell arteritis; Non |
| | | alcoholic steatohepatitis; Takayasu |
| | | disease; Myocardial Infarction; Alcohol |
| | | liver injury; Paracetamol liver injury; Stroke |
| 3 | P672_RHIPU | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer |
| | | disease; Myocarditis; Giant Cell |
| | | Myocarditis; Primary biliary |
| | | cirrhosis; ANCA Vasculitis; Giant cell |
| | | arteritis; Non alcoholic |
| | | steatohepatitis; Takayasu |
| | | disease; Myocardial Infarction; Alcohol |
| | | liver injury; Paracetamol liver injury; Stroke |
| 4 | P698_RHISA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Alzheimer disease; Chagas |
| | | Myocarditis; Giant Cell Myocarditis; Heart |
| | | allograft rejection; ANCA Vasculitis; Giant |
| | | cell arteritis; Non alcoholic |
| | | steatohepatitis; Takayasu |
| | | disease; Myocardial Infarction; Paracetamol |
| | | liver injury |
| 5 | P943_IXORI | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Psoriasis; Alzheimer disease; Chagas |
| | | Myocarditis; Giant Cell Myocarditis; Heart |
| | | allograft rejection; ANCA Vasculitis; Giant |
| | | cell arteritis; Non alcoholic |
| | | steatohepatitis; Takayasu |
| | | disease; Myocardial Infarction; Alcohol |
| | | liver injury; Paracetamol liver injury; Stroke |
| 6 | P974_AMBCA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer disease; Chagas |
| | | Myocarditis; Myocarditis; Giant Cell |
| | | Myocarditis; Heart allograft |
| | | rejection; Primary biliary cirrhosis; ANCA |
| | | Vasculitis; Giant cell arteritis; Non |
| | | alcoholic steatohepatitis; Takayasu |
| | | disease; Myocardial Infarction; Alcohol |
| | | liver injury; Paracetamol liver injury; Stroke |
| 7 | P983_AMBCA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer |
| | | disease; Myocarditis; Giant Cell |
| | | Myocarditis; Primary biliary |
| | | cirrhosis; ANCA Vasculitis; Non alcoholic |
| | | steatohepatitis; Takayasu |
| | | disease; Myocardial Infarction; Alcohol |
| | | liver injury; Paracetamol liver injury; Stroke |
| 8 | P985_AMBPA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer disease; Chagas |
| | | Myocarditis; Myocarditis; Giant Cell |
| | | Myocarditis; Heart allograft |
| | | rejection; Primary biliary |
| | | cirrhosis; Autoimmune hepatitis; ANCA |
| | | Vasculitis; Giant cell arteritis; Non |
| | | alcoholic steatohepatitis; Takayasu |
| | | disease; Myocardial Infarction; Alcohol |
| | | liver injury; Paracetamol liver injury; Stroke |
| 9 | P991_AMBCA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer disease; Chagas |
| | | Myocarditis; Myocarditis; Giant Cell |
| | | Myocarditis; Heart allograft |
| | | rejection; Primary biliary cirrhosis; Primary |
| | | sclerosing cholangitis; Autoimmune |
| | | hepatitis; ANCA Vasculitis; Giant cell |
| | | arteritis; Non alcoholic |
| | | steatohepatitis; Takayasu |
| | | disease; Myocardial Infarction; Alcohol |
| | | liver injury; Paracetamol liver injury; Stroke |
| 10 | P993_AMBPA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer disease; Chagas |
| | | Myocarditis; Giant Cell Myocarditis; Heart |
| | | allograft |
| | | rejection; Myocarditis_enteroviral; Primary |
| | | biliary cirrhosis; ANCA Vasculitis; Giant |
| | | cell arteritis; Non alcoholic |
| | | steatohepatitis; Takayasu |
| | | disease; Myocardial Infarction; Alcohol |
| | | liver injury; Paracetamol liver injury; Stroke |
| 11 | P1005_AMBMA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Psoriasis; Alzheimer disease; Chagas |
| | | Myocarditis; Giant Cell Myocarditis; Heart |
| | | allograft |
| | | rejection; Myocarditis_enteroviral; Primary |
| | | biliary cirrhosis; ANCA Vasculitis; Giant |
| | | cell arteritis; Non alcoholic |
| | | steatohepatitis; Takayasu |
| | | disease; Myocardial Infarction; Alcohol |
| | | liver injury; Paracetamol liver injury; Stroke |
| 12 | P1006_AMBCA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Alzheimer disease; Chagas |
| | | Myocarditis; Giant Cell Myocarditis; Heart |
| | | allograft rejection; Primary sclerosing |
| | | cholangitis; ANCA Vasculitis; Giant cell |
| | | arteritis; Non alcoholic |
| | | steatohepatitis; Takayasu |
| | | disease; Myocardial Infarction; Paracetamol |
| | | liver injury |
| 13 | P1011_AMBTR | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Alzheimer disease; Chagas |
| | | Myocarditis; Giant Cell Myocarditis; Heart |
| | | allograft rejection; Primary sclerosing |
| | | cholangitis; ANCA Vasculitis; Giant cell |
| | | arteritis; Non alcoholic |
| | | steatohepatitis; Takayasu |
| | | disease; Myocardial Infarction; Paracetamol |
| | | liver injury |
| 14 | P1014_AMBCA | Atherosclerosis; Inflammatory bowel |
| | | disease; Idiopathic pulmonary |
| | | fibrosis; Multiple sclerosis; Breast |
| | | cancer; Psoriasis; ANCA |
| | | Vasculitis; Myocardial Infarction |
| 15 | P1015_AMBCA | Atherosclerosis; Inflammatory bowel |
| | | disease; Rheumatoid arthritis; Idiopathic |
| | | pulmonary fibrosis; Multiple |
| | | sclerosis; Breast cancer; Colorectal |
| | | cancer; Psoriasis; Myocarditis; Giant Cell |
| | | Myocarditis; Primary biliary |
| | | cirrhosis; Primary sclerosing |
| | | cholangitis; Autoimmune hepatitis; ANCA |
| | | Vasculitis; Giant cell arteritis; Alcohol liver |
| | | injury; Stroke |
| 16 | P1030_AMBPA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer disease; Chagas |
| | | Myocarditis; Giant Cell Myocarditis; Heart |
| | | allograft |
| | | rejection; Myocarditis_enteroviral; Primary |
| | | biliary cirrhosis; ANCA Vasculitis; Giant |
| | | cell arteritis; Non alcoholic |
| | | steatohepatitis; Takayasu |
| | | disease; Myocardial Infarction; Alcohol |
| | | liver injury; Paracetamol liver injury; Stroke |
| 17 | P1063_AMBTR | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Alzheimer disease; Chagas |
| | | Myocarditis; Giant Cell Myocarditis; Heart |
| | | allograft rejection; ANCA Vasculitis; Giant |
| | | cell arteritis; Non alcoholic |
| | | steatohepatitis; Takayasu |
| | | disease; Myocardial Infarction; Paracetamol |
| | | liver injury |
| 18 | P1146_IXORI | Atherosclerosis; Inflammatory bowel |
| | | disease; Rheumatoid arthritis; Idiopathic |
| | | pulmonary fibrosis; Skin fibrosis; Breast |
| | | cancer; Psoriasis; Alzheimer |
| | | disease; Myocarditis_enteroviral; Primary |
| | | biliary cirrhosis; ANCA Vasculitis; Non |
| | | alcoholic steatohepatitis; Takayasu |
| | | disease; Myocardial Infarction; Alcohol |
| | | liver injury; Paracetamol liver injury; Stroke |
| 19 | P1156_IXORI | Atherosclerosis; Inflammatory bowel |
| | | disease; Rheumatoid arthritis; Liver |
| | | fibrosis; Idiopathic pulmonary fibrosis; Skin |
| | | fibrosis; Breast cancer; Psoriasis; Alzheimer |
| | | disease; Myocarditis_enteroviral; Primary |
| | | biliary cirrhosis; ANCA Vasculitis; Non |
| | | alcoholic steatohepatitis; Takayasu |
| | | disease; Myocardial Infarction; Alcohol |
| | | liver injury; Paracetamol liver injury; Stroke |
| 20 | P1180_AMBTR | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer |
| | | disease; Myocarditis; Giant Cell |
| | | Myocarditis; Primary biliary |
| | | cirrhosis; ANCA Vasculitis; Giant cell |
| | | arteritis; Non alcoholic |
| | | steatohepatitis; Takayasu |
| | | disease; Myocardial Infarction; Alcohol |
| | | liver injury; Paracetamol liver injury; Stroke |
| 21 | P1181_AMBMA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer |
| | | disease; Myocarditis; Giant Cell |
| | | Myocarditis; Primary biliary |
| | | cirrhosis; ANCA Vasculitis; Giant cell |
| | | arteritis; Non alcoholic |
| | | steatohepatitis; Takayasu |
| | | disease; Myocardial Infarction; Alcohol |
| | | liver injury; Paracetamol liver injury; Stroke |
| 22 | P1182_AMBMA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Psoriasis; Alzheimer |
| | | disease; Myocarditis; Giant Cell |
| | | Myocarditis; Primary biliary |
| | | cirrhosis; ANCA Vasculitis; Giant cell |
| | | arteritis; Non alcoholic |
| | | steatohepatitis; Takayasu |
| | | disease; Myocardial Infarction; Alcohol |
| | | liver injury; Paracetamol liver injury; Stroke |
| 23 | P1183_AMBTR | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer |
| | | disease; Myocarditis; Giant Cell |
| | | Myocarditis; Primary biliary |
| | | cirrhosis; ANCA Vasculitis; Non alcoholic |
| | | steatohepatitis; Takayasu |
| | | disease; Myocardial Infarction; Alcohol |
| | | liver injury; Paracetamol liver injury; Stroke |
| 24 | P1209_AMBPA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer disease; Chagas |
| | | Myocarditis; Myocarditis; Giant Cell |
| | | Myocarditis; Heart allograft |
| | | rejection; Myocarditis_enteroviral; Primary |
| | | biliary cirrhosis; Primary sclerosing |
| | | cholangitis; Autoimmune hepatitis; ANCA |
| | | Vasculitis; Giant cell arteritis; Non |
| | | alcoholic steatohepatitis; Takayasu |
| | | disease; Myocardial Infarction; Alcohol |
| | | liver injury; Paracetamol liver injury; Stroke |
| 25 | P1215_AMBPA | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal cancer; Alzheimer |
| | | disease; Chagas |
| | | Myocarditis; Myocarditis; Giant Cell |
| | | Myocarditis; Heart allograft |
| | | rejection; Primary biliary cirrhosis; Primary |
| | | sclerosing cholangitis; Autoimmune |
| | | hepatitis; ANCA Vasculitis; Giant cell |
| | | arteritis; Non alcoholic |
| | | steatohepatitis; Takayasu |
| | | disease; Myocardial Infarction; Paracetamol |
| | | liver injury |
| 26 | P1219_IXORI | Atherosclerosis; Inflammatory bowel |
| | | disease; Rheumatoid arthritis; Liver |
| | | fibrosis; Idiopathic pulmonary |
| | | fibrosis; Multiple |
| | | sclerosis; Psoriasis; Alzheimer |
| | | disease; Chagas Myocarditis; Heart allograft |
| | | rejection; Primary biliary |
| | | cirrhosis; Autoimmune hepatitis; ANCA |
| | | Vasculitis; Giant cell arteritis; Non |
| | | alcoholic steatohepatitis; Myocardial |
| | | Infarction; Alcohol liver injury |
| 27 | P1220_IXORI | Atherosclerosis; Inflammatory bowel |
| | | disease; Primary sclerosing cholangitis |
| 28 | P1224_IXORI | Atherosclerosis; Inflammatory bowel |
| | | disease; Rheumatoid arthritis; Liver |
| | | fibrosis; Idiopathic pulmonary |
| | | fibrosis; Multiple |
| | | sclerosis; Psoriasis; Alzheimer |
| | | disease; Chagas Myocarditis; Heart allograft |
| | | rejection; Primary biliary |
| | | cirrhosis; Autoimmune hepatitis; ANCA |
| | | Vasculitis; Giant cell arteritis; Non |
| | | alcoholic steatohepatitis; Myocardial |
| | | Infarction; Alcohol liver injury |
| 29 | P1243_AMBAM | Atherosclerosis; Inflammatory bowel |
| | | disease; Rheumatoid arthritis; Atopic |
| | | Dermatitis; Liver fibrosis; Idiopathic |
| | | pulmonary fibrosis; Kidney fibrosis; Skin |
| | | fibrosis; Multiple sclerosis; Breast |
| | | cancer; Colorectal |
| | | cancer; Psoriasis; Alzheimer disease; Chagas |
| | | Myocarditis; Giant Cell Myocarditis; Heart |
| | | allograft rejection; Primary biliary |
| | | cirrhosis; ANCA Vasculitis; Giant cell |
| | | arteritis; Non alcoholic |
| | | steatohepatitis; Takayasu |
| | | disease; Myocardial Infarction; Alcohol |
| | | liver injury; Paracetamol liver injury; Stroke |
| 30 | P1252_AMBAM | Atherosclerosis; Inflammatory bowel |
| | | disease; Rheumatoid arthritis; Atopic |
| | | Dermatitis; Idiopathic pulmonary |
| | | fibrosis; Skin fibrosis; Breast |
| | | cancer; Colorectal cancer; Psoriasis; Chagas |
| | | Myocarditis; Giant Cell |
| | | Myocarditis; Primary biliary |
| | | cirrhosis; ANCA Vasculitis; Giant cell |
| | | arteritis; Alcohol liver injury; Stroke |
| 31 | P1283_IXORI | Atherosclerosis; Inflammatory bowel |
| | | disease; Rheumatoid arthritis; Liver |
| | | fibrosis; Idiopathic pulmonary |
| | | fibrosis; Multiple |
| | | sclerosis; Psoriasis; Alzheimer |
| | | disease; Chagas Myocarditis; Heart allograft |
| | | rejection; Primary biliary |
| | | cirrhosis; Autoimmune hepatitis; ANCA |
| | | Vasculitis; Giant cell arteritis; Non |
| | | alcoholic steatohepatitis; Myocardial |
| | | Infarction; Alcohol liver injury |

### Polypeptides of the Invention

The invention provides a polypeptide comprising (a) an amino acid sequence shown in any one of SEQ ID NOs: 1 to 31 or (b) an amino acid sequence having at least 70% homology or amino identity to a sequence of (a) over its entire length. The terms “polypeptide” and “protein” may be used interchangeably herein. In (a), the polypeptide comprises an amino acid sequence shown in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31.

The polypeptide may comprise (a) all or part of an amino acid sequence shown in any one of SEQ ID NOs 1-3, 6-10, 15-16, 20-23 and 29-30; or (b) all or part of an amino acid sequence having at least 70% homology or amino identity to a sequence of (a) over its entire length. Polypeptides selected from this group (also termed Class I Evasins herein) bind one or more human chemokines selected from CCL2, CCL13 and/or CCL20. The polypeptide typically also binds at least one additional human chemokine. The polypeptide may also bind one or more of, including all of CCL8, CCL7 and CCL3L1. The polypeptide may also additionally or alternatively bind CCL13. The polypeptide preferably inhibits the one or more chemokines/additional chemokines described above. The polypeptide may be derived as defined in (a) and (b) above from one of SEQ ID NOs 1, 9 and 29.

The polypeptide may alternatively comprise (a) all or part of an amino acid sequence shown in any one of SEQ ID NOs 5, 19, 24-26, 28 and 31; or (b) all or part of an amino acid sequence having at least 70% homology or amino identity to a sequence of (a) over its entire length. Polypeptides selected from this group (also termed Class II Evasins herein) bind one or more human chemokines selected from CXCL3, CXCL10 and/or CXCL12. The polypeptide typically also binds at least one additional human chemokine. The additional human chemokine may be one or both of CXCL1 and CXCL8. The polypeptide preferably inhibits the one or more chemokines/additional chemokines described above.

The polypeptide may alternatively comprise (a) all or part of an amino acid sequence shown in any one of SEQ ID NOs 4, 11-14, 17-18 and 27; or (b) all or part of an amino acid sequence having at least 70% homology or amino identity to a sequence of (a) over its entire length. Polypeptides selected from this group (also termed Class III Evasins herein) display alternative chemokine binding characteristics to the polypeptides of the two groups described above.

The polypeptide may be derived from a tick species which infects humans.

The polypeptide can be any length. The polypeptide is preferably at least 40 amino acids in length, such as at least 50, at least 60, at least 70 or at least 80 amino acids in length. The polypeptide is preferably 250 amino acids or fewer in length, such as 200 amino acids or fewer, 150 amino acids or fewer or 100 amino acids or fewer in length. The length of the polypeptide typically depends on the length of any one of SEQ ID NOs 1 to 31. Deletions and/or extension are allowable in accordance with the invention as discussed in detail below. The polypeptide is typically from 40 to 250 amino acids in length, such as from 45 to 200 amino acids in length or from 50 to 160 amino acids in length.

The polypeptide is typically formed from naturally-occurring amino acids. The polypeptide may contain non-naturally-occurring amino acids. The polypeptide typically comprises L-amino acids. The polypeptide may comprise D-amino acids.

A variant of any one of SEQ ID NOs: 1 to 31 is a polypeptide that has an amino acid sequence which varies from that of any one of SEQ ID NOs: 1 to 31 and has the ability to bind to and inhibit one or more chemokines. A variant of any one of SEQ ID NOs: 1 to 31 is a polypeptide that has an amino acid sequence which varies from that of any one of SEQ ID NOs: 1 to 31 and has the ability to inhibit one or more chemokines.

The variant preferably binds and preferably inhibits to the same chemokines as the sequence on which the variant is based. For instance, a variant of SEQ ID NO: 1 is a polypeptide that has an amino acid sequence which varies from that of SEQ ID NO: 1 and has the ability to bind to the chemokines shown in column C or column D of SEQ ID NO: l's row in Table 2. A variant of SEQ ID NO: 1 is preferably a polypeptide that has an amino acid sequence which varies from that of SEQ ID NO: 1 and has the ability to inhibit the chemokines shown in column C or column D of SEQ ID NO: l's row in Table 2. The same is true for any of SEQ ID NOs: 2 to 31. Thus, variants of the Class II and Class III Evasins as described above preferably bind to and preferably inhibit the same chemokines as the sequence on which the variant is based,

The ability of a variant to bind to and preferably inhibit a chemokine can be assayed using any method known in the art. Suitable methods are described in the Examples and Figures, and include yeast surface display and biolayer interferometry (for binding) and chemotaxis assays (for inhibition).

The variant may be a naturally occurring variant which is expressed naturally, for instance in ticks. Alternatively, the variant may be expressed in vitro or recombinantly as discussed below. Variants also include non-naturally occurring variants produced by recombinant technology.

Over the entire length of the amino acid sequence of any one of SEQ ID NOs: 1 to 31, a variant will preferably be at least 70% homologous or identical to that sequence. More preferably, the variant may have at least 75%, at least 80%, at least 85%, at least 90% and more preferably at least 95%, 97% or 99% homology or amino acid identity to the amino acid sequence of any one of SEQ ID NOs: 1 to 31 over the entire sequence. There may be at least 80%, for example at least 85%, 90% or 95%, homology or amino acid identity over a stretch of 20 or more, for example 30, 40, 50, 60, 70, or more, contiguous amino acids (“hard homology” or “hard identity”).

Standard methods in the art may be used to determine homology. For example the UWGCG Package provides the BESTFIT program, which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

Amino acid substitutions may be made to the amino acid sequences of SEQ ID NOs: 1 to 31, for example up to 1, 2, 3, 4, 5, 10, 20, 30 or 50 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 6 below. Where amino acids have similar polarity, this can also be determined by reference to the hydropathy scale for amino acid side chains in Table 7.

TABLE 6
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 7
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acids of the amino acid sequence of any one of SEQ ID NOs: 1 to 31 may additionally be deleted from the polypeptides described above. Up to 1, 2, 3, 4, 5, 10, 20 or 30 amino acids may be deleted, or more.

Variants may include fragments of any one of SEQ ID NOs: 1 to 31. Such fragments typically retain the chemokine-binding domain of any one of SEQ ID NOs: 1 to 31. Fragments may be at least 40, at least 50, at least 60, at least 70, at least 80, at least 90 or at least 100 amino acids in length.

One or more amino acids may be alternatively or additionally added to the polypeptides described above. Put another way, the polypeptide may comprise a sequence consisting of any one of SEQ ID NOs: 1 to 31 or variant thereof having an N-terminal and/or C-terminal extension of a number of amino acids. The N-terminal and/or C-terminal extension may comprise 1, 2, 3, 4, 5, 6, 7, 8, 9 or 10 amino acids or more, such as 15, 20, 30, 40, 50 or 100 amino acids.

The invention encompasses any pharmaceutically acceptable salt of a polypeptide described herein. Said pharmaceutically acceptable salts include, for example, mineral acid salts such as chlorides, hydrochlorides, hydrobromides, phosphates, sulfates, and the like; and the salts of organic acids such as acetates, propionates, malonates, benzoates, and the like; and salts of monocationic metal ions such as sodium and potassium and the like; and salts of bases such as ammonia. A hydrochloride salt or an acetate salt is preferred.

The polypeptide may be labelled with a detectable label. The detectable label may be any suitable label which allows the polypeptide to be detected. Suitable labels include, but are not limited to, fluorescent molecules, radioisotopes, e.g. ¹²⁵I, ³⁵S, enzymes, antibodies, antigens, polynucleotides and ligands such as biotin. The label is preferably a tracer that is suitable for positron emission tomography (PET), such as fluorine (¹⁸F). The label is preferably a tracer suitable for magnetic resonance imaging (MRI), such as fluorine (¹⁹F).

The polypeptides of the invention may be made in any way. They may be made in accordance with the invention as discussed in more detail below.

The polypeptides described herein can be prepared by any suitable technique.

Alternatively, the polypeptide may be made by solid-phase peptide synthesis (SPPS) is a preferred technique. This involves formation of the peptide on small solid beads. Using SPPS, the polypeptide remains covalently attached to a bead during synthesis. The polypeptide is synthesised using repeated cycles of coupling-washing-deprotection-washing. In particular, the free N-terminal amine of a solid-phase attached polypeptide is coupled to a single N-protected amino acid unit. This unit is then deprotected, revealing a new N-terminal amine to which a further protected amino acid is attached. These steps are repeated until the polypeptide is complete. The polypeptide is then cleaved from the beads using a suitable reagent.

Suitable protecting groups, reagents, solvents and reaction conditions for SPPS are well known to those skilled in the art and as such conditions can be determined by one skilled in the art by routine optimization procedures.

Pharmaceutically acceptable salts of polypeptides can be prepared by any suitable technique. Typically, salification involves reaction of the polypeptide or a salt thereof with a suitable reagent, typically acid, to obtain the pharmaceutically acceptable salt selected.

For example, a hydrochloride salt of a polypeptide can be prepared by initially cleaving the polypeptide from the solid phase using trifluoroacetic acid. The polypeptide will thus initially be a trifluoroacetate salt. The trifluoroacetate salt can then be converted into a hydrochloride salt by any known technique, such as ion exchange on a suitable column using hydrochloric acid as an eluent.

The polypeptide or polypeptide salt products can be purified, where required, by any suitable technique. High pressure liquid chromatography (HPLC) can be used, for example. The term “polypeptide” includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immunol. 159, 3230-3237. This approach involves making pseudopolypeptides containing changes involving the backbone, and not the orientation of side chains. Meziere et al (1997) show that, at least for MHC class-II and T helper cell responses, these pseudopolypeptides are useful. Retro-inverse polypeptides, which contain NH—CO bonds instead of CO—NH peptide bonds, are much more resistant to proteolysis. Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N- or C-terminus so as to help reduce susceptibility to exoproteolytic digestion. For example, the N-terminal amino group of the polypeptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH₂→—NH(Me) or —N(Me)₂).

Polypeptides according to the invention may also include peptide variants that increase or decrease the polypeptide's half-life in vivo. Examples of analogues capable of increasing the half-life of polypeptides used according to the invention include peptoid analogues of the peptides, D-amino acid derivatives of the peptides, and peptide-peptoid hybrids. A further embodiment of the variant polypeptides used according to the invention comprises D-amino acid forms of the polypeptide. The preparation of polypeptides using D-amino acids rather than L-amino acids greatly decreases any unwanted breakdown of such an agent by normal metabolic processes, decreasing the amounts of agent which needs to be administered, along with the frequency of its administration.

The polypeptides may also be derived from amino acid mutants, glycosylation variants and other covalent derivatives of the parent allergen polypeptides. Exemplary derivatives include molecules wherein the polypeptides of the invention are covalently modified by substitution, chemical, enzymatic, or other appropriate means with a moiety other than a naturally occurring amino acid. Further included are naturally occurring variant amino acid sequences of the parent polypeptides. Such a variant amino acid sequence may be encoded by an allelic variant or represent an alternative splicing variant.

Modifications as described above may be prepared during synthesis of the peptide or by post-production modification, or when the polypeptide is in recombinant form using the known techniques of site-directed mutagenesis, random mutagenesis, or enzymatic cleavage and/or ligation of nucleic acids.

The polypeptides described herein may also be modified to improve physicochemical characteristics. Thus, for example, original amino acid sequences may be altered to improve their solubility, and accordingly a polypeptide of the invention having a variant sequence will preferably be more soluble than a polypeptide having the corresponding original amino acid sequence under equivalent conditions. Methods for evaluating the solubility of polypeptides are well known in the art.

Improved solubility is advantageous for the treatment of subjects in accordance with the invention, since administration of poorly soluble agents to subjects causes undesirable inflammatory responses. The solubility of the polypeptides may be improved by altering the residues which flank the region containing a T cell epitope. For example, N- and C-terminal to the residues of the polypeptide which flank a T cell epitope, at least one amino acid may be added selected from arginine, lysine, histidine, glutamate and aspartate. In other examples:

i) any hydrophobic residues in the up to three amino acids at the N- or C-terminus of the native sequence of the polypeptide, which are not comprised in a T cell epitope, are deleted; and/or

ii) any two consecutive amino acids comprising the sequence Asp-Gly in the up to four amino acids at the N- or C-terminus of the native sequence of the polypeptide, which are not comprised in a T cell epitope, are deleted; and/or

iii) one or more positively charged residues are added at the N and/or C terminus of the native sequence of the polypeptide.

Optionally, any polypeptides containing cysteine residues may be engineered to prevent dimer formation such that any cysteine residues are replaced with serine or 2-aminobutyric acid.

### Fusion Polypeptides of the Invention

The present invention also provides a fusion polypeptide comprising fusion polypeptide comprising a polypeptide of the invention linked to a second peptide or polypeptide. The polypeptide of the invention may be any of those discussed above.

The polypeptide of the invention is typically covalently linked to the second peptide or polypeptide. The polypeptide of the invention is typically genetically fused to the second peptide or polypeptide. The polypeptide of the invention is genetically fused to the second peptide or polypeptide if the whole construct is expressed from a single polynucleotide sequence. The coding sequences of the polypeptide of the invention and the second peptide or polypeptide may be combined in any way to form a single polynucleotide sequence encoding the construct. They may be genetically fused in any configuration. They are typically fused via their terminal amino acids. For instance, the amino terminus of the polypeptide of the invention may be fused to the carboxy terminus of the second peptide or polypeptide and vice versa.

The polypeptide of the invention may be attached directly to the second peptide or polypeptide. The polypeptide of the invention is preferably attached to the second peptide or polypeptide using one or more linkers. The one or more linkers may be designed to constrain the mobility of the polypeptides. Suitable linkers include, but are not limited to, chemical crosslinkers and peptide linkers. Peptide linker are preferred if the polypeptide of the invention and second peptide or polypeptide are genetically fused. Preferred linkers are amino acid sequences (i.e. peptide linkers). The length, flexibility and hydrophilicity of the peptide linker are typically designed such that it does not to disturb the functions of the polypeptide of the invention. Preferred flexible peptide linkers are stretches of 2 to 20, such as 4, 6, 8, 10 or 16, serine and/or glycine amino acids. More preferred flexible linkers include (SG)1, (SG)2, (SG)3, (SG)4, (SG)5 and (SG)8 wherein S is serine and G is glycine. Preferred rigid linkers are stretches of 2 to 30, such as 4, 6, 8, 16 or 24, proline amino acids. More preferred rigid linkers include (P)12 wherein P is proline.

The polypeptide of the invention may be transiently attached to the second peptide or polypeptide by a hex-his tag or Ni-NTA. They may also be modified such that they transiently attach to each other. The polypeptide of the invention may also be attached to the second peptide or polypeptide via cysteine linkage. This can be mediated by a bi-functional chemical linker or by a polypeptide linker with a terminal presented cysteine residue.

The second peptide or polypeptide may be any peptide or protein. The second protein is preferably a fragment crystallizable region (Fc region). The Fc region may be from any of the types of subject discussed below. Fc region is preferably human. The Fc region may derived from any isotype of antibody, such as IgA, IgD, IgG, IgE or IgM.

The second peptide or polypeptide may be an epitope tag or purification tag or cell-surface display tag or a tag that enables or facilitates systemic peptide delivery or delivery and targeting to a specific organ or to a tumour, or facilitates transfer across a barrier such as skin or gut or blood brain barrier. Suitable tags are known in the art. Suitable tags include, but are not limited to, AviTag, calmodulin-tag, polyglutamate tag, E-tag, FLAG-tag, HA-tag, His-tag, Myc-tag, S-tag, SBP-tag, Softag 1, Softag 3, Strep-tag, TC tag, V5 tag, VSV-tag, Xpress tag, Isopeptag, SpyTag, SnoopTag, BCCP (Biotin Carboxyl Carrier Protein), Glutathione-S-transferase-tag, Green fluorescent protein-tag, Halo-tag, Maltose binding protein-tag, Nus-tag, Thioredoxin-tag, Strep-tag, Skin permeating and cell entering (SPACE)-tag, TD1-tag, magainin tag, TAT-tag, penetratin-tag, cell penetrating peptide (CPP)-tag, Fc tag. The second peptide or polypeptide may be a signal petode, such as an IgK peptide.

The fusion polypeptide may be labelled with a detectable label. The detectable label may be any of those discussed above.

### Polypeptide Combinations of the Invention

The invention also provides a combination of two or more polypeptides of the invention, i.e. two or more different polypeptides of the invention. The combination may comprise two or more polypeptides of the invention, two or more fusion polypeptides of the invention or a two or more of both types of polypeptide. The combination may comprise two or more different polypeptides comprising different variants of the same Evasin, i.e. two or more different variants of any one of SEQ ID NOs: 1 to 31. Such combinations may further comprise a polypeptide comprising any one of SEQ ID NOs: 1 to 31.

The combination may comprise any number of different polypeptides of the invention. For instance, the combination may comprise 3, 4, 5, 6, 7, 8, 9, 10, 11, 12, 13, 14, 15, 16, 17, 18, 19, 20, 21, 22, 23, 24, 25, 26, 27, 28, 29, 30 or 31 different polypeptides of the invention. The combination may comprise 10 or more, 20 or more, 30 or more, 40 or more or 50 or more polypeptides of the invention.

The combination preferably comprises different polypeptides of the invention which inhibit the same one or more chemokine(s) as shown in Table 2. For instance, the combination may comprise a polypeptide comprising SEQ ID NO: 5 or a variant thereof and a polypeptide comprising SEQ ID NO: 17 or a variant thereof. The skilled person can design other suitable combinations.

One or more of, such as all of, the polypeptides in the combination may be labelled with a detectable label. The label may be any of those discussed above. Different polypeptides in the combination may be labelled with the same detectable label or different detectable labels.

### Polynucleotides of the Invention

The invention also provides a polynucleotide which encodes a polypeptide of the invention. The polypeptide may be any of those discussed above.

The invention also provides a polynucleotide which encodes a fusion polypeptide of the invention. The fusion polypeptide is preferably genetically fused as discussed above.

The invention also provides a polynucleotide which encodes a combination of the invention. The coding sequences for the two or more polypeptides in the combination may be present in a single polynucleotide of the invention. This is typically the case when the combination is encoded by a single vector of the invention.

A polynucleotide, such as a nucleic acid, is a polymer comprising two or more nucleotides. The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hydroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light.

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide may be single stranded or double stranded.

The polynucleotide sequence encodes the relevant polypeptide(s) on the basis of the genetic code, including its degeneracy.

Polynucleotide sequences may be derived and replicated using standard methods in the art, for example using PCR involving specific primers. It is straightforward to generate polynucleotide sequences using such standard techniques. These are discussed in more detail below.

### Polynucleotide Combinations of the Invention

The invention also provides a combination of two or more polynucleotides each of which encodes a polypeptide of the invention, i.e. each of which encodes a different polypeptide of the invention. The combination may encode two or more polypeptides of the invention, two or more fusion polypeptides of the invention or a two or more of both types of polypeptide. The combination may encode two or more different polypeptides comprising different variants of the same Evasin, i.e. two or more different variants of any one of SEQ ID NOs: 1 to 31. Such combinations may further comprise a polynucleotide encoding a polypeptide comprising any one of SEQ ID NOs: 1 to 31.

The combination may comprise any number of different polynucleotide. For instance, the combination may comprise 3, 4, 5, 6, 7, 8, 9, 10, 11, 12, 13, 14, 15, 16, 17, 18, 19, 20, 21, 22, 23, 24, 25, 26, 27, 28, 29, 30 or 31 different polynucleotide of the invention. The combination may comprise 10 or more, 20 or more, 30 or more, 40 or more or 50 or more polynucleotide of the invention.

The combination preferably comprises two or more polynucleotides which encode different polypeptides of the invention which inhibit the same one or more chemokine(s) as shown in Table 2. For instance, the combination may comprise a polynucleotide encoding a polypeptide comprising SEQ ID NO: 5 or a variant thereof and a polynucleotide encoding a polypeptide comprising SEQ ID NO: 17 or a variant thereof. The skilled person can design other suitable combinations.

### Vectors of the Invention

The invention also provides a vector comprising a polynucleotide of the invention or a combination of two or more polynucleotides of the invention.

The vector may be a cloning vector. The amplified sequences may be incorporated into a recombinant replicable vector such as a cloning vector. The vector may be used to replicate the polynucleotide in a compatible host cell. Thus polynucleotide sequences may be made by introducing the polynucleotide into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The vector may be an expression vector. The polynucleotide sequence may be cloned into any suitable expression vector. In an expression vector, the polynucleotide of the invention or the combination of the invention is typically operably linked to a control sequence which is capable of providing for the expression of the polynucleotide or the combination by the host cell. Such expression vectors can be used to express one or more polypeptides of the invention.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The term “control sequence” is intended to include promoters, enhancers, internal ribosomal entry sites (IRES), and other expression control elements (e.g. transcription termination signals, such as polyadenylation signals and poly-U sequences). Such control sequences are described, for example, in Goeddel, GENE EXPRESSION TECHNOLOGY: METHODS IN ENZYMOLOGY 185, Academic Press, San Diego, Calif. (1990). Control sequences include those that direct constitutive expression of a nucleotide sequence in many types of brain cell and those that direct expression of the nucleotide sequence only in certain brain cells. A non-limiting example of a suitable neuron-specific promoters include the neurofilament promoter; Byrne and Ruddle, 1989. Proc. Natl. Acad. Sci. USA 86: 5473-5477.

Control sequences may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “control sequence” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit Q-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. With regards to control sequences, mention is made of U.S. patent application Ser. No. 10/491,026. With regards to promoters, mention is made of PCT publication WO 2011/028929 and U.S. application Ser. No. 12/511,940.

The expression vector may then be introduced into a suitable host cell. Thus, polypeptide of the invention can be produced by inserting a polynucleotide or a combination into an expression vector, introducing the vector into a compatible bacterial host cell, and growing the host cell under conditions which bring about expression of the polynucleotide or combination. The vectors may be for example, plasmid, virus or phage vectors provided with an origin of replication, optionally a promoter for the expression of the said polynucleotide or combination and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or X_{L} promoter is typically used.

The vector may be used to administer a polynucleotide of the invention or a combination of two or more polynucleotides to a subject as discussed in more detail below. Conventional viral and non-viral based gene transfer methods can be used to introduce the polynucleotide or combination into cells. Non-viral vector delivery systems include DNA plasmids, RNA, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Conventional viral based expression systems could include retroviral, lentivirus, adenoviral, adeno-associated (AAV) and herpes simplex virus (HSV) vectors for gene transfer. Methods for producing and purifying such vectors are know in the art.

Exemplary vector systems for using the invention are a virus, such as rAAV, that comprises or consists essentially of an exogenous polynucleotide encoding the polypeptide, fusion polypeptide or polypeptide combination of the invention, e.g., a cassette comprising or consisting essentially of a promoter, a polynucleotide encoding the polypeptide, fusion polypeptide or polypeptide combination of the invention and a terminator.

Since AAV is a DNA virus, the polynucleotides used in AAV or rAAV are advantageously DNA.

The vector may be delivered using nanoparticle delivery systems. Such delivery systems include, but are not limited to, lipid-based systems, liposomes, micelles, microvesicles, exosomes, and gene gun. With regard to nanoparticles that can deliver RNA, see, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93. Lipid Nanoparticles, Spherical Nucleic Acid (SNA™) constructs, nanoplexes and other nanoparticles (particularly gold nanoparticles) are also contemplated as a means for delivery of a polynucleotide or a polynucleotide of the invention. The invention provides any of these deliver systems comprising a vector of the invention, a polynucleotide of the invention or a polynucleotide combination of the invention.

In some embodiments, the vector may form a component of an inducible system. The inducible nature of the system would allow for spatiotemporal control of expression of a polypeptide of the invention or a combination of such polypeptides using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc), or light inducible systems (Phytochrome, LOV domains, or cryptochrome).

As will be clear from below, the polynucleotide of the invention or a polynucleotide combination of the invention or any expression vector containing these components may be present in a population of cells. The cells may be administered to the subject. Suitable ways of modifying and administering cells are known in the art.

### Host Cells of the Invention

The invention also provides a host cell which comprises a polynucleotide of the invention, a polynucleotide combination of the invention or a vector of the invention. The host cell may be used to replicate the polynucleotide, combination or vector. The host cell may be used to express a polypeptide of the invention or a combination of polypeptides of the invention in vitro. The host cell may be used to deliver the polynucleotide, combination or vector to a subject in need thereof as discussed below.

Host cells will be chosen to be compatible with the cloning or expression vector used to transform the cell. Suitable conditions are known in the art (see, for instance, Sambrook, J. and Russell, D. supra).

Suitable cells for use in the invention include prokaryotic cells and eukaryotic cells. The prokaryotic cell is preferably a bacterial cell. Suitable bacterial cells include, but are not limited to, Escherichia coli, Corynebacterium and Pseudomonas fluorescens. Any E. coli cell with a DE3 lysogen, for example C41 (DE3), BL21 (DE3), JM109 (DE3), B834 (DE3), TUNER, Origami and Origami B, can express a vector comprising the T7 promoter.

Suitable eukaryotic cells include, but are not limited to, Saccharomyces cerevisiae, Pichia pastoris, filamentous fungi, such as Aspergillus, Trichoderma and Myceliophthora thermophila C1, baculovirus-infected insect cells, such as Sf9, Sf21 and High Five strains, non-lytic insect cells, Leishmania cells, plant cells, such as tobacco plant cells, and mammalian cells, such as Bos primigenius cells (Bovine), Mus musculus cells (Mouse), Chinese Hamster Ovary (CHO) cells, Human Embryonic Kidney (HEK) cells, Baby Hamster Kidney (BHK) cells and HeLa cells. Other preferred mammalian cells include, but are not limited to, PC12, HEK293, HEK293A, HEK293T, CHO, BHK-21, HeLa, ARPE-19, RAW264.7 and COS cells.

The host cell is preferably HEK293T.

If the cell is being administered to a subject, the cell is preferably derived from the subject or a subject of the same species. For instance, a human cell is typically administered to a human subject. The host cell is preferably autologous. In other words, the cell is preferably derived from the subject into which the cell will be administered. Alternatively, the host cell is preferably allogeneic. In other words, the cell is preferably derived from a patient that is immunologically compatible with the patient into which the cell will be administered.

The cell may be isolated, substantially isolated, purified or substantially purified. The cell is isolated or purified if it is completely free of any other components, such as culture medium or other cell types. The cell is substantially isolated if it is mixed with carriers or diluents, such as culture medium and others discussed above and below, which will not interfere with its intended use. Alternatively, the host cell of the invention may be present in a growth matrix or immobilized on a surface as discussed below.

### Pharmaceutical Compositions of the Invention

The invention also provides a pharmaceutical composition comprising (a) a polypeptide of the invention, a polypeptide combination of the invention, a polynucleotide of the invention, a vector of the invention or a host cell of the invention and (b) a pharmaceutically acceptable carrier or diluent. The carrier or diluent may be any of those discussed above with reference to the vectors of the invention.

The carrier(s) or diluent(s) present in the pharmaceutical composition must be “acceptable” in the sense of being compatible with the other ingredients of the composition and not deleterious to the recipient thereof. Typically, carriers for injection, and the final formulation, are sterile and pyrogen free. Preferably, the carrier or diluent is water. A pharmaceutically acceptable carrier or diluent may comprise as one of its components thioglycerol or thioanisole.

Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

The active agents are typically present at 0.1% to 50% by weight in the pharmaceutical composition, more preferably at 0.1% to 5% by weight. They may be present at less than 0.1% by weight in the pharmaceutical composition.

The pharmaceutically acceptable carrier or diluent is typically present at 50% to 99.9% by weight in the pharmaceutical composition, more preferably at 95% to 99.9% by weight. The pharmaceutically acceptable carrier or diluents may be present at more than 99.9% by weight in the pharmaceutical composition.

Pharmaceutical compositions include, but are not limited to pharmaceutically acceptable solutions, lyophilisates, suspensions, emulsions in oily or aqueous vehicles, pastes, and implantable sustained-release or biodegradable compositions. Such pharmaceutical compositions may further comprise one or more additional ingredients including, but not limited to, suspending, stabilizing, or dispersing agents. A lyophilisate may comprise one or more of trehalose, thioglycerol and thioanisole. In one embodiment of a pharmaceutical composition for parenteral administration, the active ingredient is provided in dry form (e.g., a lyophilisate, powder or granules) for reconstitution with a suitable vehicle (e.g., sterile pyrogen-free water) prior to parenteral administration of the reconstituted pharmaceutical composition.

The pharmaceutical composition may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable compositions may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parenterally-administrable pharmaceutical compositions which are useful include those which comprise the active ingredient in microcrystalline form, in a liposomal preparation, or as a component of a biodegradable polymer systems. Pharmaceutical compositions for sustained release or implantation may comprise pharmaceutically acceptable polymeric or hydrophobic materials such as an emulsion, an ion exchange resin, a sparingly soluble polymer, or a sparingly soluble salt.

For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical compositions. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Oral compositions include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release compositions or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to an individual may be provided with an enteric coating comprising, for example, Eudragit “S”, Eudragit “L”, cellulose acetate, cellulose acetate phthalate or hydroxypropylmethyl cellulose.

Polynucleotides may be present in combination with cationic lipids, polymers or targeting systems.

Uptake of polynucleotide or oligonucleotide constructs may be enhanced by several known transfection techniques, for example those including the use of transfection agents. Examples of these agents include cationic agents, for example, calcium phosphate and DEAE-Dextran and lipofectants, for example, lipofectamine and transfectam. The dosage of the polynucleotide or oligonucleotide to be administered can be altered.

Alternatively, the active agent may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules. The composition will depend upon factors such as the nature of the active agent and the method of delivery. The pharmaceutical composition may be administered in a variety of dosage forms. It may be administered orally (e.g. as tablets, troches, lozenges, aqueous or oily suspensions, dispersible powders or granules), topically, parenterally, subcutaneously, by inhalation, intravenously, intramuscularly, intralymphatically (such as to lymph nodes in the groin), intrasternally, transdermally, intradermally, epidermally, sublingually, intranasally, buccally or by infusion techniques. The administration may be intratonsillar. The administration may be as suppositories. The administration may be made by iontophoresis. Preferably, the administration is intradermal, epidermal or transdermal. The administration may be made by a patch, such as a microtine patch. Administration is discussed in more detail below.

A physician will be able to determine the required route and means of administration for each particular individual.

The pharmaceutical compositions of the invention are preferably provided sealed in a container. The pharmaceutical compositions are typically provided in unit dose form, for example single dose form. They may alternatively be provided in multi-dose form. Where the pharmaceutical composition is a pharmaceutically acceptable solution, the solution may be provided in an ampoule, sealed vial, syringe, cartridge, flexible bag or glass bottle. Where the pharmaceutical composition is a lyophilisate, it is preferably provided in a sealed vial.

The pharmaceutical compositions of the invention will comprise a suitable concentration of each agent to be effective without causing adverse reaction. Where the pharmaceutical composition is for example a lyophilisate, the relevant concentration will be that of each polypeptide following reconstitution. Typically, the concentration of each agent in the pharmaceutical composition when in solution will be in the range of 0.03 to 200 nmol/ml. The concentration of each agent may be more preferably in the range of 0.3 to 200 nmol/ml, 3 to 180 nmol/ml, 5 to 160 nmol/ml, 10 to 150 nmol/ml, 50 to 200 nmol/ml or 30 to 120 nmol/ml, for example about 100 nmol/ml. The pharmaceutical composition should have a purity of greater than 95% or 98% or a purity of at least 99%.

In an embodiment where the invention involves combines therapy, the other therapeutic agents or adjuvants may be administered separately, simultaneously or sequentially. They may be administered in the same or different pharmaceutical compositions. A pharmaceutical composition may therefore be prepared which comprises an agent of the invention and also one or more other therapeutic agents or adjuvants. A pharmaceutical composition of the invention may alternatively be used simultaneously, sequentially or separately with one or more other therapeutic compositions as part of a combined treatment.

### In Vitro Methods of the Invention

The invention also provides a method of inhibiting the signalling of one or more chemokines in an in vitro culture, the method comprising contacting the culture with a polypeptide of the invention, a combination of the invention, a polynucleotide of the invention, a vector of the invention or a host cell of the invention.

The method may comprise inhibiting any number of chemokines, such as 1, 2, 3, 4, 5, 6, 7, 8, 9, 10, 11, 12, 13 or 14 chemokines. The chemokines may be selected from any of those in Table 2. The one or more chemokines are preferably identified in column C and/or column D of a particular row of Table 2. When inhibiting the one or more chemokines in a particular row in Table 3, a polypeptide, combination, polynucleotide, vector or host cell of the invention based on the Evasin in the same row is preferably used in the method of the invention. For instance, when inhibiting CCL8, a polypeptide, combination, polynucleotide, vector or host cell of the invention based on the sequence shown in SEQ ID NO: 8 is preferably used. Similarly, when inhibiting CCL2 or CCL1/CCL2/CCL3/CCL5, a polypeptide, combination, polynucleotide, vector or host cell of the invention based on the sequence shown in SEQ ID NO: 1 is preferably used. When inhibiting one or more of CCL2, CCl3 and/or CCL20, a polypeptide, combination, polynucleotide, vector or host cell of the invention based on the sequence shown in any one of SEQ ID NOs 1-3, 6-10, 15-16, 20-23 and 29-30 is preferably used. When inhibiting one or more of CXCL3, CXCL10 and/or CXCL12, a polypeptide, combination, polynucleotide, vector or host cell of the invention based on the sequence shown in any one of SEQ ID NOs 5, 19, 24-26, 28 and 31 is preferably used.

The in vitro culture is preferable a culture of cells capable of undergoing chemotaxis. The in vitro culture is preferably a chemotactic assay. The culture may be present in a culture flask or the wells of a flat plate, such as a standard 96 or 384 well plate. Such plates are commercially available Fisher scientific, VWR suppliers, Nunc, Starstedt or Falcon. Conditions for culturing cells are known in the art.

The polypeptide, combination, polynucleotide, vector or host cell of the invention may be administered in any of the forms discussed above.

### Therapeutic Methods of the Invention

The invention also provides a method of inhibiting the signalling of one or more chemokines in a subject, the method comprising administering to the subject a polypeptide of the invention, a combination of the invention, a polynucleotide of the invention, a vector of the invention or a host cell of the invention. The invention also provides a polypeptide of the invention, a combination of the invention, a polynucleotide of the invention, a vector of the invention or a host cell of the invention for use in a method of inhibiting the signalling of one or more chemokines in a subject. The invention also provides use of a polypeptide of the invention, a combination of the invention, a polynucleotide of the invention, a vector of the invention or a host cell of the invention in the manufacture of a medicament for use in inhibiting the signalling of one or more chemokines in a subject.

The method may comprise inhibiting any number of chemokines, such as 1, 2, 3, 4, 5, 6, 7, 8, 9, 10, 11, 12, 13 or 14 chemokines. The chemokines may be selected from any of those in Table 2. The one or more chemokines are preferably identified in column C and/or column D of a particular row of Table 2. When inhibiting the one or more chemokines in a row of Table 2, a polypeptide, combination, polynucleotide, vector or host cell of the invention based on the Evasin in the same row is preferably used in the method of the invention. For instance, when inhibiting CCL8, a polypeptide, combination, polynucleotide, vector or host cell of the invention based on the sequence shown in SEQ ID NO: 8 may be used. Similarly, when inhibiting CCL2 or CCL1/CCL2/CCL3/CCL5, a polypeptide, combination, polynucleotide, vector or host cell of the invention based on the sequence shown in SEQ ID NO: 1 may be used. When inhibiting one or more of CCL2, CCl3 and/or CCL20, a polypeptide, combination, polynucleotide, vector or host cell of the invention based on the sequence shown in any one of SEQ ID NOs 1-3, 6-10, 15-16, 20-23 and 29-30 is preferably used. When inhibiting one or more of CXCL3, CXCL10 and/or CXCL12, a polypeptide, combination, polynucleotide, vector or host cell of the invention based on the sequence shown in any one of SEQ ID NOs 5, 19, 24-26, 28 and 31 is preferably used.

The skilled person can design combinations of Evasins to inhibit specific combinations of chemokines.

The invention also provides a method of treating or preventing in a subject one or more diseases associated with one or more chemokines, the method comprising administering to the subject a polypeptide of the invention, a combination of the invention, a polynucleotide of the invention, a vector of the invention or a host cell of the invention. The invention also provides a polypeptide of the invention, a combination of the invention, a polynucleotide of the invention, a vector of the invention or a host cell of the invention for use in a method of treating or preventing in a subject one or more diseases associated with one or more chemokines. The invention also provides use of a polypeptide of the invention, a combination of the invention, a polynucleotide of the invention, a vector of the invention or a host cell of the invention in the manufacture of a medicament for treating or preventing in a subject one or more diseases associated with one or more chemokines.

A disease is associated with one or more chemokines if the disease has a chemokine component. In other words, one or more symptoms of the disease may be treated or prevented by inhibiting one or more chemokines. Any number of chemokines may be involved as discussed above. The chemokines are preferably selected from those shown in Table 2.

The method may comprise treating or preventing any number of diseases associated with one or more chemokines, such as 1, 2, 3, 4, 5, 6, 7, 8, 9, 10, 11, 12 or 13 diseases. The chemokines may be selected from any of those in Table 2. The one or more diseases are preferably identified in column 3 of a particular row of Table 4 or 5. When treating or preventing the one or more diseases in a particular row of Table 4 or 5, a polypeptide, combination, polynucleotide, vector or host cell of the invention based on the Evasin in the same row is preferably used in the method of the invention.

As seen from FIG. 5 and Tables 4 and 5, diseases that may be treated or prevented by Class I evasins (polypeptides of the invention based on SEQ ID NOs 1-3, 6-10, 15-16, 20-23 and 29-30 as described above, and related polynucleotides/combinations/host cells) include diseases where CCL2 is known to be expressed including myocarditis, myocardial infarction, skin fibrosis, ANCA vasculitis, Takayasu disease, Atherosclerosis, stroke, multiple sclerosis, Alzheimer disease, primary biliary cirrhosis, liver fibrosis, non alcoholic steato hepatitis, paracetamol liver injury, alcohol liver injury, idiopathic pulmonary fibrosis, kidney fibrosis, inflammatory bowel disease, rheumatoid arthritis, and breast cancer; where CCL13 is known to be expressed, including giant cell myocarditis, myocardial infarction, ANCA vasculitis, atherosclerosis, idiopathic pulmonary fibrosis, and rheumatoid arthritis, and where CCL20 is known to be expressed including giant cell myocarditis, ANCA vasculitis, giant cell arteritis, atherosclerosis, stroke, primary biliary cirrhosis, alcohol liver injury, idiopathic pulmonary fibrosis, inflammatory bowel disease, rheumatoid arthritis, psoriasis, breast cancer and colorectal cancer.

Diseases that may be treated or prevented by Class II evasins (polypeptides of the invention based on SEQ ID NOs 5, 19, 24-26, 28 and 31 as described above, and related polynucleotides/combinations/host cells) include diseases where CXCL3 is known to be expressed, including, idiopathic pulmonary fibrosis and breast cancer, where CXCL10 is known to be expressed, including atherosclerosis, inflammatory bowel disease, rheumatoid arthritis, liver fibrosis, idiopathic pulmonary fibrosis, multiple sclerosis, psoriasis, alzheimer disease, chagas myocarditis, heart allograft rejection, primary biliary cirrhosis, autoimmune hepatitis, ANCA vasculitis, giant cell arteritis, non alcoholic steatohepatitis, myocardial infarction, and alcohol liver injury, or where CXCL12 is expressed, as in atherosclerosis, inflammatory bowel disease, rheumatoid arthritis, idiopathic pulmonary fibrosis, multiple sclerosis, colorectal cancer, myocarditis, primary biliary cirrhosis, primary sclerosing cholangitis, and autoimmune hepatitis.

Preferably the disease comprises expression of more than one chemokine, such as two or more, three or more, four or more, or five or more chemokines. The disease may be an inflammatory disease comprising expression of more than one chemokine, such as two or more, three or more, four or more, or five or more chemokines. The disease to be treated or prevented may comprise expression of one or more, such as up to two, three or four chemokines not bound by previously described Evasins (data for binding of previously described Evasins shown in FIG. 3). The disease may comprise expression of one or more of CCL2, CCL13 and/or CCL20, but not of other chemokines bound by previously described Evasins as shown in FIG. 3. The disease may comprise expression of one or more of CXCL3, CXCL10 and/or CXCL12, but not expression of other chemokines bound by previously described Evasins as shown in FIG. 3. The disease may not comprise expression of any chemokines bound by previously described Evasins. The above definitions of disease are also applicable to diseases to be prognosed or diagnosed in accordance with the invention, as discussed below.

For instance, when treating or preventing RA, a polypeptide, combination, polynucleotide, vector or host cell of the invention based on the sequence shown in SEQ ID NO: 3 may be used. Similarly, when treating or preventing one or more of atherosclerosis, RA, IBD, liver fibrosis, lung fibrosis, kidney fibrosis, skin fibrosis, multiple sclerosis, breast cancer, or Alzheimer's disease, a polypeptide, combination, polynucleotide, vector or host cell of the invention based on the sequence shown in SEQ ID NO: 1 may be used.

Preferably, the disease to be treated or prevented is myocarditis, giant cell myocarditis, myocardial infarction, stroke or idiopathic pulmonary fibrosis. A polypeptide, combination, polynucleotide, vector or host cell of the invention based on the sequence shown in SEQ ID NO: 29 or the sequence shown in SEQ ID NO: 9 may preferably be used for treatment or prevention of the above diseases. A polypeptide, combination, polynucleotide, vector or host cell of the invention based on the sequence shown in SEQ ID NO: 1 may also be used for treatment or prevention of the above diseases.

The skilled person can design combinations of Evasins to treat or prevent specific combinations of diseases.

Any subject may be treated in accordance with the invention. The subject is typically human. However, the subject can be another animal or mammal, such as a research animal, such as a rat, a mouse, a rabbit or a guinea pig, a commercially farmed animal, such as a horse, a cow, a sheep or a pig, or a pet, such as a cat, a dog or a hamster.

The subject may be asymptomatic. A prophylactically effective amount of the polypeptide, combination, polynucleotide, vector or host cell is administered to such a subject. A prophylactically effective amount is an amount which prevents the onset of one or more, preferably all of, symptoms of the one or more diseases.

Alternatively, the subject may be in need thereof. That is, the subject may exhibit one or more symptoms of the one or more diseases. A therapeutically effective amount of the polypeptide, combination, polynucleotide, vector or host cell is administered to such an subject. A therapeutically effective amount is an amount which is effective to ameliorate one or more of, preferably all of, the symptoms of the one or more diseases.

The polypeptide, combination, polynucleotide, vector or host cell may be administered to the subject in any appropriate way. In the invention, the polypeptide, combination, polynucleotide, vector or host cell may be administered in a variety of dosage forms. Thus, it can be administered orally, for example as tablets, troches, lozenges, aqueous or oily suspensions, dispersible powders or granules. It may also be administered by enteral or parenteral routes such as via buccal, anal, pulmonary, intravenous, intra-arterial, intramuscular, intraperitoneal, intraarticular, topical or other appropriate administration routes. A physician will be able to determine the required route of administration for each particular subject.

The polypeptide, combination, polynucleotide, vector or host cell may be in any of the forms discussed above with reference to the pharmaceutical composition of the invention.

Methods for gene delivery are known in the art. See, e.g., U.S. Pat. Nos. 5,399,346, 5,580,859 and 5,589,466. The nucleic acid molecule can be introduced directly into the recipient subject, such as by standard intramuscular or intradermal injection; transdermal particle delivery; inhalation; topically, or by oral, intranasal or mucosal modes of administration. The molecule alternatively can be introduced ex vivo into cells that have been removed from a subject. For example, a polynucleotide, expression cassette or vector of the invention may be introduced into APCs of an individual ex vivo. Cells containing the nucleic acid molecule of interest are re-introduced into the subject such that an immune response can be mounted against the peptide encoded by the nucleic acid molecule. The nucleic acid molecules used in such immunization are generally referred to herein as “nucleic acid vaccines.”

The dose may be determined according to various parameters, especially according to the substance used; the age, weight and condition of the subject to be treated; the route of administration; and the required regimen. Again, a physician will be able to determine the required route of administration and dosage for any particular subject. A typical daily dose is from about 0.1 to 50 mg per kg of body weight, according to the activity of the specific inhibitor, the age, weight and conditions of the subject to be treated and the frequency and route of administration. The dose may be provided as a single dose or may be provided as multiple doses, for example taken at regular intervals, for example 2, 3 or 4 doses administered hourly. Preferably, dosage levels of inhibitors are from 5 mg to 2 g.

Typically polynucleotide or oligonucleotide inhibitors are administered in the range of 1 pg to 1 mg, preferably to 1 pg to 10 pg nucleic acid for particle mediated delivery and 10 μg to 1 mg for other routes.

The polypeptide, the combination, the polynucleotide, the vector or the host cell is preferably administered in combination with another therapy

The inhibitor may be used in combination with one or more other therapies intended to treat the same subject. By a combination is meant that the therapies may be administered simultaneously, in a combined or separate form, to the subject. The therapies may be administered separately or sequentially to a subject as part of the same therapeutic regimen. For example, the polypeptide, the combination, the polynucleotide, the vector or the host cell be used in combination with another therapy intended to treat the one or more disease. The other therapy may be a general therapy aimed at treating or improving the condition of the subject. For example, treatment with methotrexate, glucocorticoids, salicylates, nonsteroidal anti-inflammatory drugs (NSAIDs), analgesics, other DMARDs, aminosalicylates, corticosteroids, and/or immunomodulatory agents (e.g., 6-mercaptopurine and azathioprine) may be combined with the inhibitor. The other therapy may be a specific treatment directed at the one or more diseases. Such treatments are known in the art. For instance in the treatment of rheumatoid arthritis this may include anti-TNFα [121] or other biologics targeting other cytokines (e.g. IL7, IL17, IL17) or their receptors (e.g. IL1-R, IL-6R), that are in clinical use or development[122]. In the treatment of inflammatory bowel disease we may use biologics such as vedolizumab [123]. For atherosclerosis we may use simvastatin or other statins.

### Antibodies of the Invention

The invention also provides an antibody or a fragment thereof which specifically binds a polypeptide comprising (a) an amino acid sequence shown in any one of SEQ ID NOs: 1 to 31 or (b) an amino acid sequence having at least 70% homology or amino identity to a sequence of (a) over its entire length. The antibody or fragment thereof preferably specifically binds a polypeptide comprising an amino acid sequence shown in any one of SEQ ID NOs: 1 to 31.

An antibody “specifically binds” to a polypeptide when it binds with preferential or high affinity to that polypeptide but does not substantially bind, does not bind or binds with only low affinity to other polypeptides. For instance, an antibody “specifically binds” to SEQ ID NO: 1 or a variant thereof when it binds with preferential or high affinity to SEQ ID NO: 1 or a variant thereof but does not substantially bind, does not bind or binds with only low affinity to other polypeptides. The same applies to any one of SEQ ID NOs: 2 to 31.

An antibody binds with preferential or high affinity if it binds with a Kd of 1×10-7 M or less, more preferably 5×10-8 M or less, more preferably 1×10-8 M or less or more preferably 5×10-9 M or less. An antibody binds with low affinity if it binds with a Kd of 1×10-6 M or more, more preferably 1×10-5 M or more, more preferably 1×10-4 M or more, more preferably 1×10-3 M or more, even more preferably 1×10-2 M or more. A variety of protocols for competitive binding or immunoradiometric assays to determine the specific binding capability of compounds, such as antibodies or antibody constructs and oligonucleotides are well known in the art (see for example Maddox et al, J. Exp. Med. 158, 1211-1226, 1993).

The antibody may be, for example, a monoclonal antibody, a polyclonal antibody, a single chain antibody, a chimeric antibody, a CDR-grafted antibody or a humanized antibody. The antibody may be an intact immunoglobulin molecule or a fragment thereof such as a Fab, F(ab′)₂ or Fv fragment. Furthermore, the antibodies and fragment thereof may be chimeric antibodies, CDR-grafted antibodies or humanised antibodies.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising an antibody in a host animal against the whole polypeptide or a fragment thereof, for example an antigenic epitope thereof, hereinafter the “immunogen”. The fragment may be any of the fragments mentioned herein (typically at least 10 or at least 15 amino acids long).

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified. A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

### Diagnostic Methods of the Invention

The invention also provides a method of detecting one or more chemokines in a tissue, comprising contacting the tissue with a detectably-labelled polypeptide of the invention or a detectably-labelled polypeptide combination of the invention and detecting the binding of the polypeptide or the combination to one or more chemokines in the tissue. The polypeptide may be a fusion polypeptide of the invention. The tissue may be in vitro or in vivo. The invention also provides a detectably-labelled polypeptide of the invention or a detectably-labelled combination of the invention for use in a method of detecting one or more chemokines in a tissue. The invention also provides use of a detectably-labelled polypeptide of the invention or a detectably-labelled combination in the manufacture of medicament for detecting one or more chemokines in a tissue.

Any method of detecting binding may be used. The method may be positron emission tomography (PET) or magnetic resonance imaging (MRI).

The tissue may be any tissue. The tissue is preferably in a subject. The subject may be any those discussed above. The polypeptide or combination may be administered to the subject in any of the forms discussed above.

Any of the polypeptides of the invention or combinations of the invention discussed above may be used. Suitable detectable labels are also discussed above. The label is preferably a tracer that is suitable for positron emission tomography (PET), such as fluorodeoxyglucose (¹⁸F). The label is preferably a tracer suitable for magnetic resonance imaging (MRI), such as fluorine (¹⁹F).

The method may comprise detecting any number of chemokines, such as 1, 2, 3, 4, 5, 6, 7, 8, 9, 10, 11, 12, 13 or 14 chemokines. The chemokines may be selected from any of those in Table 2. The one or more chemokines are preferably identified in column C and/or column D of a particular row of Table 2. When detecting the one or more chemokines in a row of Table 2, a polypeptide or combination of the invention based on the Evasin in the same row is preferably used in the method of the invention. For instance, when detecting CCL8, a polypeptide, combination, polynucleotide, vector or host cell of the invention based on the sequence shown in SEQ ID NO: 8 may be used. Similarly, when detecting CCL2 or CCL1/CCL2/CCL3/CCL5, a polypeptide, combination, polynucleotide, vector or host cell of the invention based on the sequence shown in SEQ ID NO: 1 may be used. The skilled person can design combinations of Evasins to detect specific combinations of chemokines.

The method is preferably for diagnosing or prognosing inflammation, one or more diseases associated with one or more chemokines or one or more diseases identified in column 3 of a particular row of Table 4 or 5.

The method may comprise diagnosing or prognosing any number of diseases associated with one or more chemokines, such as 1, 2, 3, 4, 5, 6, 7, 8, 9, 10, 11, 12 or 13 diseases. The chemokines may be selected from any of those in Table 2. The one or more diseases are preferably identified in column 3 of a particular row of Table 4 or 5. When diagnosing or prognosing the one or more diseases in a particular row of Table 4 or 5, a polypeptide, combination, polynucleotide, vector or host cell of the invention based on the Evasin in the same row is preferably used in the method of the invention.

For instance, when diagnosing or prognosing RA, a polypeptide, combination, polynucleotide, vector or host cell of the invention based on the sequence shown in SEQ ID NO: 3 may be used. Similarly, when diagnosing or prognosing one or more of atherosclerosis, RA, IBD, liver fibrosis, lung fibrosis, kidney fibrosis, skin fibrosis, multiple sclerosis, breast cancer, or Alzheimer's disease, a polypeptide, combination, polynucleotide, vector or host cell of the invention based on the sequence shown in SEQ ID NO: 1 may be used. Preferably, the disease to be diagnosed or prognosed is myocarditis, giant cell myocarditis, myocardial infarction, stroke or idiopathic pulmonary fibrosis. A polypeptide, combination, polynucleotide, vector or host cell of the invention based on the sequence shown in SEQ ID NO: 29 or the sequence shown in SEQ ID NO: 9 may preferably be used for diagnosis or prognosis of the above diseases. A polypeptide, combination, polynucleotide, vector or host cell of the invention based on the sequence shown in SEQ ID NO: 1 may also be used for diagnosis or prognosis of the above diseases.

The skilled person can design combinations of Evasins to diagnose or prognose specific combinations of chemokines.

### Examples

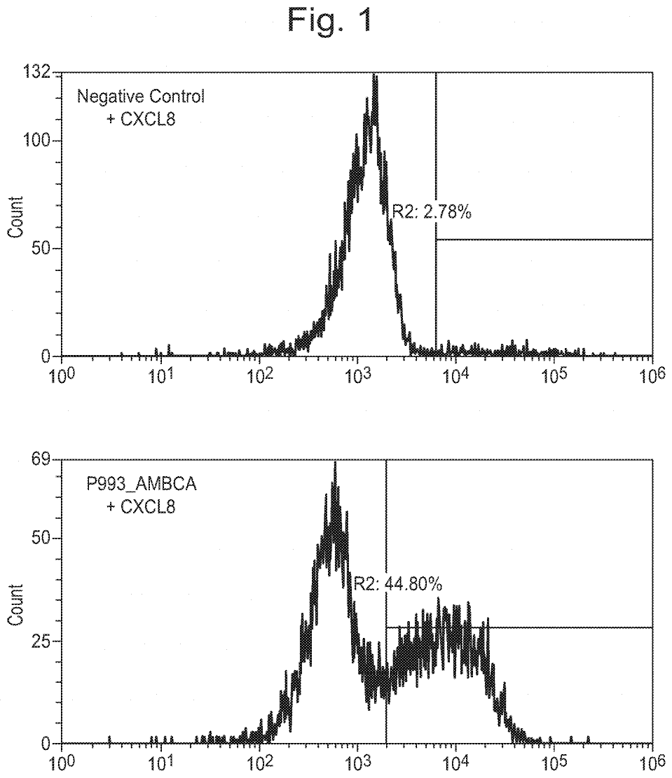

1. Identification of chemokine-binding evasins. To identify protein-protein interactions between extracellular proteins we adapted yeast surface display technology, originally developed for the identification of single chain antibodies[54]. Here candidate proteins are expressed in yeast and displayed on the cell wall. Fluorescent-activated cell sorting (FACS) is used to select a desired yeast cell that bind a fluorescent-labelled target[124]. To identify novel chemokine-binding Evasins we created yeast surface display libraries that express mature peptides identified in tick salivary transcriptomes. We systematically screened the above libraries using the chemokines CCL1, CCL2, CCL3, CCL4, CCL5, CCL8, CCL11, CCL15, CCL17, CCL18, CCL19, CCL20, CCL22, CCL25, CX3CL1, CXCL8, CXCL10, CXCL11, CXCL12, CXCL13. We have obtained 31 interacting clones to date that have been retested and binding to one or more chemokines confirmed using FACS (SEQ ID NOs: 1 to 31; Table 1, FIG. 1). This method can be used also to alter affinities and binding characteristics of an evasin e.g. through mutagenesis and FACS selection.

2. Characterisation of evasin binding to chemokines. We have used Microscale Thermophoresis (Table 3). Alternative methods are surface plasmon resonance and biolayer interferometry.

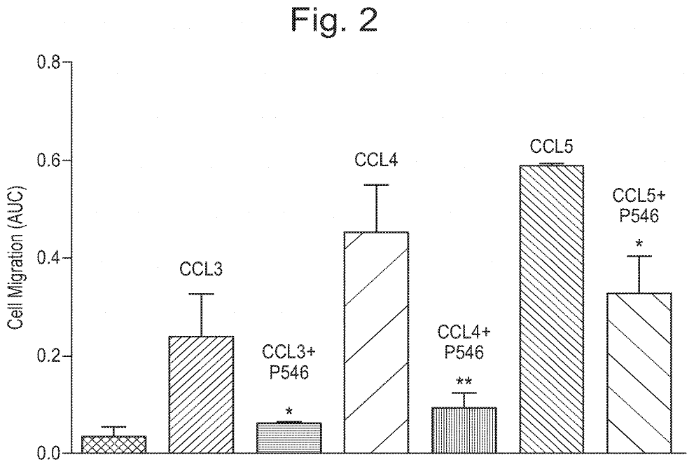

3. Characterisation of inhibition of chemokine function by an evasin. We have used real time chemotaxis using an IncucyteZOOM® system, and U2-OS cells stably transfected with CCR5. Results are shown in FIG. 2. Alternative methods include using other cell types, and end-point Boyden chamber transwell assays, or real-time ECIS chemotaxis assays.

4. Additional characterisation of evasin binding to human chemokines. Further characterisation of binding of certain novel evasins against all known human chemokines (with exception of CCL25, CCL26, CXCL16, CXCL17, CXCL4L1, XCL2) was carried out using biolayer interferometry. The data for their binding properties are shown in FIG. 3, alongside published Kd data in relation to binding of human chemokines for previously described Evasins (Evasins 1, 3 and 4). Other binding data for the novel evasins obtained using yeast surface display is also summarised. From this data three classes of novel evasins were identified, as shown in FIG. 3. Class I novel evasins bind CC-class chemokines CCL2, CCL13 or CCL20 in addition to other CC chemokines as indicated. None of the previously described evasins are reported to have binding to CCL2, CCL13 or CCL20. Class II novel evasins bind CXC-class chemokines CXC-chemokines CXCL3, CXCL10 or CXCL12 in addition to other CXC chemokines as indicated. None of the previously described evasins are reported to have binding to CXCL3, CXCL10 or CXCL12. Class III novel evasins represent other novel sequences.

5. Additional characterisation of inhibition of chemokine functions by novel evasins. Further evaluation of the neutralisation activity of certain novel evasins against particular human cytokines was carried out using a THP1 cell migration assay, with results (IC50 data) shown in FIG. 4. The results illustrate neutralisation of function of multiple chemokines by certain novel evasins.

### REFERENCES

1. Garin A, Proudfoot A E I. Chemokines as targets for therapy. Experimental Cell Research. 2011; 317(5):602-12.
2. Kiefer F, Siekmann A F. The role of chemokines and their receptors in angiogenesis. Cell Mol Life Sci. 2011; 68(17):2811-30.
3. Sahin H, Wasmuth H E. Chemokines in tissue fibrosis. Biochim Biophys Acta. 2013; 1832(7):1041-8.
4. Zernecke A, Weber C. Chemokines in atherosclerosis: proceedings resumed. Arteriosclerosis, thrombosis, and vascular biology. 2014; 34(4):742-50.
5. Saxena A, Russo I, Frangogiannis N G. Inflammation as a therapeutic target in myocardial infarction: learning from past failures to meet future challenges. Transl Res. 2016; 167(1):152-66. doi: 10.1016/j.trsl.2015.07.002. PubMed PMID: 26241027; PubMed Central PMCID: PMCPMC4684426.
6. Leuschner F, Katus H A, Kaya Z. Autoimmune myocarditis: past, present and future. Journal of autoimmunity. 2009; 33(3-4):282-9. doi: 10.1016/j.jaut.2009.07.009. PubMed PMID: 19679447.
7. Atreya R, Neurath M F. Chemokines in Inflammatory Bowel Diseases. Dig Dis. 2010; 28(3):386-94.
8. Barnes P J. Immunology of asthma and chronic obstructive pulmonary disease. Nat Rev Immunol. 2008; 8(3):183-92.
9. Szczucinski A, Losy J. Chemokines and chemokine receptors in multiple sclerosis. Potential targets for new therapies. Acta Neurol Scand. 2007; 115(3):137-46.
10. Mabuchi T, Chang T W, Quinter S, Hwang S T. Chemokine receptors in the pathogenesis and therapy of psoriasis. J Dermatol Sci. 2012; 65(1):4-11.
11. Pivarcsi A, Homey B. Chemokine networks in atopic dermatitis: traffic signals of disease. Curr Allergy Asthma Rep. 2005; 5(4):284-90.
12. Villeda S A, Luo J, Mosher K I, Zou B, Britschgi M, Bieri G, et al. The ageing systemic milieu negatively regulates neurogenesis and cognitive function. Nature. 2011; 477(7362):90-4.
13. Sarvaiya P J, Guo D, Ulasov I, Gabikian P, Lesniak M S. Chemokines in tumor progression and metastasis. Oncotarget. 2013; 4(12):2171-85. PubMed PMID: 24259307; PubMed Central PMCID: PMCPMC3926818.
14. Zlotnik A, Burkhardt A M, Homey B. Homeostatic chemokine receptors and organ-specific metastasis. Nat Rev Immunol. 2011; 11(9):597-606. doi: 10.1038/nri3049. PubMed PMID: 21866172.
15. Schall T J, Proudfoot A E I. Overcoming hurdles in developing successful drugs targeting chemokine receptors. Nature Publishing Group. 2011; 11(5):355-63.
16. Zlotnik A, Yoshie O, Nomiyama H. The chemokine and chemokine receptor superfamilies and their molecular evolution. Genome Biology. 2006; 7(12):243.
17. DeVries M E, Kelvin A A, Xu L, Ran L, Robinson J, Kelvin D J. Defining the origins and evolution of the chemokine/chemokine receptor system. J Immunol. 2006; 176(1):401-15.
18. Bachelerie F, Ben-Baruch A, Burkhardt A M, Combadiere C, Farber J M, Graham G J, et al. International Union of Basic and Clinical Pharmacology. [corrected]. LXXXIX. Update on the extended family of chemokine receptors and introducing a new nomenclature for atypical chemokine receptors. Pharmacol Rev. 2014; 66(1):1-79. doi: 10.1124/pr.113.007724. PubMed PMID: 24218476; PubMed Central PMCID: PMCPMC3880466.
19. Albert R, Jeong H, Barabasi A L. Error and attack tolerance of complex networks. Nature. 2000; 406(6794):378-82. doi: 10.1038/35019019. PubMed PMID: 10935628.
20. Combadiere C, Potteaux S, Rodero M, Simon T, Pezard A, Esposito B, et al. Combined inhibition of CCL2, CX3CR1, and CCR5 abrogates Ly6C(hi) and Ly6C(lo) monocytosis and almost abolishes atherosclerosis in hypercholesterolemic mice. Circulation. 2008; 117(13):1649-57.
21. Saederup N, Chan L, Lira S A, Charo I F. Fractalkine deficiency markedly reduces macrophage accumulation and atherosclerotic lesion formation in CCR2−/− mice: evidence for independent chemokine functions in atherogenesis. Circulation. 2008; 117(13):1642-8.
22. Mattes J, Yang M, Mahalingam S, Kuehr J, Webb D C, Simson L, et al. Intrinsic defect in T cell production of interleukin (IL)-13 in the absence of both IL-5 and eotaxin precludes the development of eosinophilia and airways hyperreactivity in experimental asthma. J Exp Med. 2002; 195(11):1433-44.
23. Mirshahpanah P, Li Y Y, Burkhardt N, Asadullah K, Zollner™. CCR4 and CCR10 ligands play additive roles in mouse contact hypersensitivity. Experimental dermatology. 2008; 17(1):30-4. doi: 10.1111/j.1600-0625.2007.00630.x. PubMed PMID: 18095942.
24. Rangel-Moreno J, Moyron-Quiroz J E, Hartson L, Kusser K, Randall T D. Pulmonary expression of CXC chemokine ligand 13, CC chemokine ligand 19, and CC chemokine ligand 21 is essential for local immunity to influenza. Proc Natl Acad Sci USA. 2007; 104(25):10577-82.
25. Moriguchi K, Miyamoto K, Tanaka N, Yoshie O, Kusunoki S. The importance of CCR4 and CCR6 in experimental autoimmune encephalomyelitis. J Neuroimmunol. 2013; 257(1-2):53-8.
26. Pope S M, Zimmermann N, Stringer K F, Karow M L, Rothenberg M E. The eotaxin chemokines and CCR3 are fundamental regulators of allergen-induced pulmonary eosinophilia. J Immunol. 2005; 175(8):5341-50.
27. Horuk R. Chemokine receptor antagonists: overcoming developmental hurdles. Nature Reviews Drug Discovery. 2009; 8(1):23-33.
28. Jeyaprakash A, Hoy M A. First divergence time estimate of spiders, scorpions, mites and ticks (subphylum: Chelicerata) inferred from mitochondrial phylogeny. Exp Appl Acarol. 2009; 47(1):1-18.
29. Hovius J W R, Levi M, Fikrig E. Salivating for knowledge: potential pharmacological agents in tick saliva. PLoS Med. 2008; 5(2):e43.
30. Kazimirovi M, Stibriniovi I. Tick salivary compounds: their role in modulation of host defences and pathogen transmission. Front Cell Infect Microbiol. 2013; 3:1-19.
31. Schoeler G B, Manweiler S A, Wikel S K. Ixodes scapularis: effects of repeated infestations with pathogen-free nymphs on macrophage and T lymphocyte cytokine responses of BALB/c and C3H/HeN mice. Exp Parasitol. 1999; 92(4):239-48. doi: 10.1006/expr.1999.4426. PubMed PMID: 10425152.
32. Wikel S K. Tick modulation of host immunity: an important factor in pathogen transmission. Int J Parasitol. 1999; 29(6):851-9. PubMed PMID: 10480722.
33. Schoeler G B, Manweiler S A, Wikel S K. Cytokine responses of C3H/HeN mice infested with Ixodes scapularis or Ixodes pacificus nymphs. Parasite Immunol. 2000; 22(1):31-40. PubMed PMID: 10607289.
34. Garcia G R, Gardinassi L G, Ribeiro J M, Anatriello E, Ferreira B R, Moreira H N S, et al. The sialotranscriptome of Amblyomma triste, Amblyomma parvum and Amblyomma cajennense ticks, uncovered by 454-based RNA-seq. Parasit Vectors. 2014; 7:430.
35. Karim S, Singh P, Ribeiro J M C. A Deep Insight into the Sialotranscriptome of the Gulf Coast Tick, Amblyomma maculatum. PLoS ONE. 2011; 6(12):e28525.
36. Hajnicki V, Kocakovi P, Slivikovi M, Slovik M, Gasperik J, Fuchsberger N, et al. Anti-interleukin-8 activity of tick salivary gland extracts. Parasite Immunol. 2001; 23(9):483-9.
37. VANCOVA I, Hajnicki V, Slovak M, Kocakovi P, PAESEN G C, Nuttall P A. Evasin-3-like anti-chemokine activity in salivary gland extracts of ixodid ticks during blood-feeding: a new target for tick control. Parasite Immunol. 2010; 32(6):460-3.
38. Hajnicki V, VANCOVA I, Kochkovi P, Slovik M, Gasperik J, Slivikovi M, et al. Manipulation of host cytokine network by ticks: a potential gateway for pathogen transmission. Parasitology. 2005; 130(Pt 3):333-42.
39. Deruaz M, Frauenschuh A, Alessandri A L, Dias J M, Coelho F M, Russo R C, et al. Ticks produce highly selective chemokine binding proteins with antiinflammatory activity. Journal of Experimental Medicine. 2008; 205(9):2019-31.
40. Frauenschuh A, Power C A, Déruaz M, Ferreira B R, Silva J S, Teixeira M M, et al. Molecular cloning and characterization of a highly selective chemokine-binding protein from the tick Rhipicephalus sanguineus. J Biol Chem. 2007; 282(37):27250-8.
41. Dias J M, Losberger C, Deruaz M, Power C A, Proudfoot A E I, Shaw J P. Structural Basis of Chemokine Sequestration by a Tick Chemokine Binding Protein: The Crystal Structure of the Complex between Evasin-1 and CCL3. PLoS ONE. 2009; 4(12):e8514.
42. Bonvin P, Dunn S M, Rousseau F, Dyer D P, Shaw J, Power C A, et al. Identification of the pharmacophore of the CC Chemokine-binding proteins Evasin-1 and -4 using phage display. Journal of Biological Chemistry. 2014.
43. Montecucco F, Lenglet S, Braunersreuther V, Pelli G, Pellieux C, Montessuit C, et al. Single Administration of the CXC Chemokine-Binding Protein Evasin-3 During Ischemia Prevents Myocardial Reperfusion Injury in Mice. Arteriosclerosis, thrombosis, and vascular biology. 2010; 30(7):1371-7.
44. Copin J-C, da Silva R F, Fraga-Silva R A, Capettini L, Quintao S, Lenglet S, et al. Treatment with Evasin-3 reduces atherosclerotic vulnerability for ischemic stroke, but not brain injury in mice. J Cereb Blood Flow Metab. 2013; 33(4):490-8.
45. Braunersreuther V, Montecucco F, Pelli G, Galan K, Proudfoot A E, Belin A, et al. Treatment with the CC chemokine-binding protein Evasin-4 improves post-infarction myocardial injury and survival in mice. Thromb Haemost. 2013; 110(4):807-25.
46. Montecucco F, Mach F, Lenglet S, Vonlaufen A, Gomes Quinderé A L, Pelli G, et al. Treatment with Evasin-3 abrogates neutrophil-mediated inflammation in mouse acute pancreatitis. Eur J Clin Invest. 2014; 44(10):940-50.
47. Russo R C, Alessandri A L, Garcia C C, Cordeiro B F, Pinho V, Cassali G D, et al. Therapeutic Effects of Evasin-1, a Chemokine Binding Protein, in Bleomycin-Induced Pulmonary Fibrosis. Am J Respir Cell Mol Biol. 2011; 45(1):72-80.
48. Castor M G M, Rezende B, Resende C B, Alessandri A L, Fagundes C T, Sousa L P, et al. The CCL3/macrophage inflammatory protein-1alpha-binding protein evasin-1 protects from graft-versus-host disease but does not modify graft-versus-leukemia in mice. The Journal of Immunology. 2010; 184(5):2646-54.
49. Klarenbeek A, Maussang D, Blanchetot C, Saunders M, van der Woning S, Smit M, et al. Targeting chemokines and chemokine receptors with antibodies. Drug Discovery Today: Technologies. 2012; 9(4):e237-e44.
50. Potzinger H, Geretti E, Brandner B, Wabitsch V, Piccinini A-M, Rek A, et al. Developing chemokine mutants with improved proteoglycan affinity and knocked-out GPCR activity as anti-inflammatory recombinant drugs. Biochem Soc Trans. 2006; 34(Pt 3):435-7.
51. Trinker M U, Kungl A J. Targeting chemokine-glycan interactions: the CellJammer® technology platform. Drug Discovery Today: Technologies. 2012; 9(4):e227-314.
52. Nelson A L. Antibody fragments: hope and hype. mabs. 2010; 2(1):77-83.
53. Fosgerau K, Hoffmann T. Peptide therapeutics: current status and future directions. Drug Discovery Today. 2014:1-7.
54. Boder E T, Wittrup K D. Yeast surface display for screening combinatorial polypeptide libraries. Nat Biotechnol. 1997; 15(6):553-7.
55. Zhao Y, Bishop B, Clay J E, Lu W, Jones M, Daenke S, et al. Automation of large scale transient protein expression in mammalian cells. J Struct Biol. 2011; 175(2):209-15.
56. Jerabek-Willemsen M, Wienken C J, Braun D, Baaske P, Duhr S. Molecular interaction studies using microscale thermophoresis. Assay and drug development technologies. 2011; 9(4):342-53. Epub 2011 Aug. 5. doi: 10.1089/adt.2011.0380. PubMed PMID: 21812660; PubMed Central PMCID: PMC3148787.
57. Seidel S A, Dijkman P M, Lea W A, van den Bogaart G, Jerabek-Willemsen M, Lazic A, et al. Microscale thermophoresis quantifies biomolecular interactions under previously challenging conditions. Methods. 2013; 59(3):301-15. Epub 2012 Dec. 29. doi: 10.1016/j.ymeth.2012.12.005. PubMed PMID: 23270813; PubMed Central PMCID: PMC3644557.
58. Haque N S, Zhang X, French D L, Li J, Poon M, Fallon J T, et al. CC chemokine I-309 is the principal monocyte chemoattractant induced by apolipoprotein(a) in human vascular endothelial cells. Circulation. 2000; 102(7):786-92.
59. Reape T J, Groot P H. Chemokines and atherosclerosis. Atherosclerosis. 1999; 147(2):213-25.
60. Haley K J, Lilly C M, Yang J H, Feng Y, Kennedy S P, Turi T G, et al. Overexpression of eotaxin and the CCR3 receptor in human atherosclerosis: using genomic technology to identify a potential novel pathway of vascular inflammation. Circulation. 2000; 102(18):2185-9.
61. Yu R, Kim C-S, Kawada T, Kwon T-W, Lim T-H, Kim Y-W, et al. Involvement of leukotactin-1, a novel CC chemokine, in human atherosclerosis. Atherosclerosis. 2004; 174(1):35-42.
62. Weber C, Meiler S, Doring Y, Koch M, Drechsler M, Megens R T A, et al. CCL17-expressing dendritic cells drive atherosclerosis by restraining regulatory T cell homeostasis in mice. J Clin Invest. 2011; 121(7):2898-910.
63. Calvayrac O, Rodriguez-Calvo R, Alonso J, Orbe J, Martin-Ventura J L, Guadall A, et al. CCL20 is increased in hypercholesterolemic subjects and is upregulated by LDL in vascular smooth muscle cells: role of NF-κB. Arteriosclerosis, thrombosis, and vascular biology. 2011; 31(11):2733-41.
64. Erbel C, Sato K, Meyer F B, Kopecky S L, Frye R L, Goronzy J J, et al. Functional profile of activated dendritic cells in unstable atherosclerotic plaque. Basic Res Cardiol. 2007; 102(2):123-32.
65. Kimura S, Tanimoto A, Wang K-Y, Shimajiri S, Guo X, Tasaki T, et al. Expression of macrophage-derived chemokine (CCL22) in atherosclerosis and regulation by histamine via the H2 receptor. Pathol Int. 2012; 62(10):675-83.
66. Kim C-S, Kang J-H, Cho H-R, Blankenship T N, Erickson K L, Kawada T, et al. Potential involvement of CCL23 in atherosclerotic lesion formation/progression by the enhancement of chemotaxis, adhesion molecule expression, and MMP-2 release from monocytes. Inflamm Res. 2011; 60(9):889-95.
67. Abd Alla J, Langer A, Elzahwy S S, Arman-Kalcek G, Streichert T, Quitterer U. Angiotensin-converting enzyme inhibition down-regulates the pro-atherogenic chemokine receptor 9 (CCR9)-chemokine ligand 25 (CCL25) axis. Journal of Biological Chemistry. 2010; 285(30):23496-505.
68. Mach F, Sauty A, Iarossi A S, Sukhova G K, Neote K, Libby P, et al. Differential expression of three T lymphocyte-activating CXC chemokines by human atheroma-associated cells. J Clin Invest. 1999; 104(8):1041-50.
69. Abi-Younes S, Sauty A, Mach F, Sukhova G K, Libby P, Luster A D. The stromal cell-derived factor-1 chemokine is a potent platelet agonist highly expressed in atherosclerotic plaques. Circ Res. 2000; 86(2):131-8.
70. Smedbakken L M, Halvorsen B, Daissormont I, Ranheim T, Michelsen A E, Skjelland M, et al. Increased levels of the homeostatic chemokine CXCL13 in human atherosclerosis—Potential role in plaque stabilization. Atherosclerosis. 2012; 224(1):266-73.
71. Minami M, Kume N, Shimaoka T, Kataoka H, Hayashida K, Akiyama Y, et al. Expression of SR-PSOX, a novel cell-surface scavenger receptor for phosphatidylserine and oxidized LDL in human atherosclerotic lesions. Arteriosclerosis, thrombosis, and vascular biology. 2001; 21(11):1796-800.
72. Zhang X, Feng X, Cai W, Liu T, Liang Z, Sun Y, et al. Chemokine CX3CL1 and its receptor CX3CR1 are associated with human atherosclerotic lesion vulnerability. Thromb Res. 2015; 135(6):1147-53.
73. Szekanecz Z, Koch A E. Successes and failures of chemokine-pathway targeting in rheumatoid arthritis. Nat Rev Rheumatol. 2015. doi: 10.1038/nrrheum.2015.157. PubMed PMID: 26607389.
74. Mazzucchelli L, Hauser C, Zgraggen K, Wagner H E, Hess M W, Laissue J A, et al. Differential in situ expression of the genes encoding the chemokines MCP-1 and RANTES in human inflammatory bowel disease. The Journal of pathology. 1996; 178(2):201-6. Epub 1996 Feb. 1. doi: 10.1002/(SICI)1096-9896(199602)178:2<201::AID-PATH440>3.0.CO; 2-4. PubMed PMID: 8683390.
75. Banks C, Bateman A, Payne R, Johnson P, Sheron N. Chemokine expression in IBD. Mucosal chemokine expression is unselectively increased in both ulcerative colitis and Crohn's disease. The Journal of pathology. 2003; 199(1):28-35. Epub 2002 Dec. 11. doi: 10.1002/path. 1245. PubMed PMID: 12474223.
76. Kotarsky K, Sitnik K M, Stenstad H, Kotarsky H, Schmidtchen A, Koslowski M, et al. A novel role for constitutively expressed epithelial-derived chemokines as antibacterial peptides in the intestinal mucosa. Mucosal immunology. 2010; 3(1):40-8. Epub 2009 Oct. 9. doi: 10.1038/mi.2009.115. PubMed PMID: 19812544.
77. Uguccioni M, Gionchetti P, Robbiani D F, Rizzello F, Peruzzo S, Campieri M, et al. Increased expression of IP-10, IL-8, MCP-1, and MCP-3 in ulcerative colitis. Am J Pathol. 1999; 155(2):331-6. Epub 1999 Aug. 6. doi: 10.1016/S0002-9440(10)65128-0. PubMed PMID: 10433925; PubMed Central PMCID: PMC1866859.
78. Ahrens R, Waddell A, Seidu L, Blanchard C, Carey R, Forbes E, et al. Intestinal macrophage/epithelial cell-derived CCL11/eotaxin-1 mediates eosinophil recruitment and function in pediatric ulcerative colitis. Journal of immunology. 2008; 181(10):7390-9. Epub 2008 Nov. 5. PubMed PMID: 18981162; PubMed Central PMCID: PMC2728352.
79. Puleston J, Cooper M, Murch S, Bid K, Makh S, Ashwood P, et al. A distinct subset of chemokines dominates the mucosal chemokine response in inflammatory bowel disease. Alimentary pharmacology & therapeutics. 2005; 21(2):109-20. Epub 2005 Feb. 1. doi: 10.1111/j.1365-2036.2004.02262.x. PubMed PMID: 15679760.
80. Papadakis K A, Prehn J, Moreno S T, Cheng L, Kouroumalis E A, Deem R, et al. CCR9-positive lymphocytes and thymus-expressed chemokine distinguish small bowel from colonic Crohn's disease. Gastroenterology. 2001; 121(2):246-54. Epub 2001 Aug. 7. PubMed PMID: 11487533.
81. Dotan I, Werner L, Vigodman S, Weiss S, Brazowski E, Maharshak N, et al. CXCL12 is a constitutive and inflammatory chemokine in the intestinal immune system. Inflammatory bowel diseases. 2010; 16(4):583-92. Epub 2009 Sep. 24. doi: 10.1002/ibd.21106. PubMed PMID: 19774645.
82. Sans M, Danese S, de la Motte C, de Souza H S, Rivera-Reyes B M, West G A, et al. Enhanced recruitment of CX3CR1+ T cells by mucosal endothelial cell-derived fractalkine in inflammatory bowel disease. Gastroenterology. 2007; 132(1):139-53. Epub 2007 Jan. 24. doi: 10.1053/j.gastro.2006.10.010. PubMed PMID: 17241867; PubMed Central PMCID: PMC2194659.
83. Reinecker H C, Loh E Y, Ringler D J, Mehta A, Rombeau J L, MacDermott R P. Monocyte-chemoattractant protein 1 gene expression in intestinal epithelial cells and inflammatory bowel disease mucosa. Gastroenterology. 1995; 108(1):40-50. Epub 1995 Jan. 1. PubMed PMID: 7806062.
84. Izzo R S, Witkon K, Chen Al, Hadjiyane C, Weinstein M I, Pellecchia C. Neutrophil-activating peptide (interleukin-8) in colonic mucosa from patients with Crohn's disease. Scandinavian journal of gastroenterology. 1993; 28(4):296-300. Epub 1993 Apr. 1. PubMed PMID: 8387693.
85. Mahida Y R, Ceska M, Effenberger F, Kurlak L, Lindley I, Hawkey C J. Enhanced synthesis of neutrophil-activating peptide-1/interleukin-8 in active ulcerative colitis. Clinical science. 1992; 82(3):273-5. Epub 1992 Mar. 1. PubMed PMID: 1312411.
86. Owczarek W, Paplinska M, Targowski T, Jahnz-Rozyk K, Paluchowska E, Kucharczyk A, et al. Analysis of eotaxin 1/CCL11, eotaxin 2/CCL24 and eotaxin 3/CCL26 expression in lesional and non-lesional skin of patients with atopic dermatitis. Cytokine. 2010; 50(2):181-5. Epub 2010 Mar. 20. doi: 10.1016/j.cyto.2010.02.016. PubMed PMID: 20236835.
87. Nomura I, Gao B, Boguniewicz M, Darst M A, Travers J B, Leung D Y. Distinct patterns of gene expression in the skin lesions of atopic dermatitis and psoriasis: a gene microarray analysis. The Journal of allergy and clinical immunology. 2003; 112(6):1195-202. Epub 2003 Dec. 6. doi: 10.1016/j.jaci.2003.08.049. PubMed PMID: 14657882.
88. Hijnen D, De Bruin-Weller M, Oosting B, Lebre C, De Jong E, Bruijnzeel-Koomen C, et al. Serum thymus and activation-regulated chemokine (TARC) and cutaneous T cell-attracting chemokine (CTACK) levels in allergic diseases: TARC and CTACK are disease-specific markers for atopic dermatitis. The Journal of allergy and clinical immunology. 2004; 113(2):334-40. Epub 2004 Feb. 10. doi: 10.1016/j.jaci.2003.12.007. PubMed PMID: 14767451.
89. Echigo T, Hasegawa M, Shimada Y, Takehara K, Sato S. Expression of fractalkine and its receptor, CX3CR1, in atopic dermatitis: possible contribution to skin inflammation. The Journal of allergy and clinical immunology. 2004; 113(5):940-8. Epub 2004 May 8. doi: 10.1016/j.jaci.2004.02.030. PubMed PMID: 15131578.
90. Park C O, Lee H J, Lee J H, Wu W H, Chang N S, Hua L, et al. Increased expression of CC chemokine ligand 18 in extrinsic atopic dermatitis patients. Experimental dermatology. 2008; 17(1):24-9. Epub 2007 Nov. 6. doi: 10.1111/j.1600-0625.2007.00634.x. PubMed PMID: 17979978.
91. Pivarcsi A, Gombert M, Dieu-Nosjean M C, Lauerma A, Kubitza R, Meller S, et al. CC chemokine ligand 18, an atopic dermatitis-associated and dendritic cell-derived chemokine, is regulated by staphylococcal products and allergen exposure. Journal of immunology. 2004; 173(9):5810-7. Epub 2004 Oct. 21. PubMed PMID: 15494534.
92. Kakinuma T, Saeki H, Tsunemi Y, Fujita H, Asano N, Mitsui H, et al. Increased serum cutaneous T cell-attracting chemokine (CCL27) levels in patients with atopic dermatitis and psoriasis vulgaris. The Journal of allergy and clinical immunology. 2003; 111(3):592-7. Epub 2003 Mar. 19. PubMed PMID: 12642842.
93. Krumbholz M, Theil D, Cepok S, Hemmer B, Kivisakk P, Ransohoff R M, et al. Chemokines in multiple sclerosis: CXCL12 and CXCL13 up-regulation is differentially linked to CNS immune cell recruitment. Brain. 2006; 129(Pt 1):200-11. doi: 10.1093/brain/awh680. PubMed PMID: 16280350.
94. Miyagishi R, Kikuchi S, Fukazawa T, Tashiro K. Macrophage inflammatory protein-1 alpha in the cerebrospinal fluid of patients with multiple sclerosis and other inflammatory neurological diseases. J Neurol Sci. 1995; 129(2):223-7. PubMed PMID: 7608739.
95. Simpson J E, Newcombe J, Cuzner M L, Woodroofe M N. Expression of the interferon-gamma-inducible chemokines IP-10 and Mig and their receptor, CXCR3, in multiple sclerosis lesions. Neuropathol Appl Neurobiol. 2000; 26(2):133-42. PubMed PMID: 10840276.
96. Ueno T, Toi M, Saji H, Muta M, Bando H, Kuroi K, et al. Significance of macrophage chemoattractant protein-1 in macrophage recruitment, angiogenesis, and survival in human breast cancer. Clin Cancer Res. 2000; 6(8):3282-9. PubMed PMID: 10955814.
97. Chavey C, Bibeau F, Gourgou-Bourgade S, Burlinchon S, Boissiere F, Laune D, et al. Oestrogen receptor negative breast cancers exhibit high cytokine content. Breast Cancer Res. 2007; 9(1):R15. doi: 10.1186/bcr1648. PubMed PMID: 17261184; PubMed Central PMCID: PMCPMC1851386.
98. Soria G, Yaal-Hahoshen N, Azenshtein E, Shina S, Leider-Trejo L, Ryvo L, et al. Concomitant expression of the chemokines RANTES and MCP-1 in human breast cancer: a basis for tumor-promoting interactions. Cytokine. 2008; 44(1):191-200. doi: 10.1016/j.cyto.2008.08.002. PubMed PMID: 18790652.
99. Chen J, Yao Y, Gong C, Yu F, Su S, Chen J, et al. CCL18 from tumor-associated macrophages promotes breast cancer metastasis via PITPNM3. Cancer Cell. 2011; 19(4):541-55. doi: 10.1016/j.ccr.2011.02.006. PubMed PMID: 21481794; PubMed Central PMCID: PMCPMC3107500.
100. Bell D, Chomarat P, Broyles D, Netto G, Harb G M, Lebecque S, et al. In breast carcinoma tissue, immature dendritic cells reside within the tumor, whereas mature dendritic cells are located in peritumoral areas. J Exp Med. 1999; 190(10):1417-26. PubMed PMID: 10562317; PubMed Central PMCID: PMCPMC2195690.
101. Bieche I, Chavey C, Andrieu C, Busson M, Vacher S, Le Corre L, et al. CXC chemokines located in the 4q21 region are up-regulated in breast cancer. Endocr Relat Cancer. 2007; 14(4):1039-52. doi: 10.1677/erc.1.01301. PubMed PMID: 18045955.
102. Luboshits G, Shina S, Kaplan O, Engelberg S, Nass D, Lifshitz-Mercer B, et al. Elevated expression of the CC chemokine regulated on activation, normal T cell expressed and secreted (RANTES) in advanced breast carcinoma. Cancer Res. 1999; 59(18):4681-7. PubMed PMID: 10493525.
103. Desurmont T, Skrypek N, Duhamel A, Jonckheere N, Millet G, Leteurtre E, et al. Overexpression of chemokine receptor CXCR2 and ligand CXCL7 in liver metastases from colon cancer is correlated to shorter disease-free and overall survival. Cancer Sci. 2015; 106(3):262-9. doi: 10.1111/cas.12603. PubMed PMID: 25580640; PubMed Central PMCID: PMCPMC4376434.
104. Kim J, Mori T, Chen S L, Amersi F F, Martinez S R, Kuo C, et al. Chemokine receptor CXCR4 expression in patients with melanoma and colorectal cancer liver metastases and the association with disease outcome. Ann Surg. 2006; 244(1):113-20. doi: 10.1097/01.sla.0000217690.65909.9c. PubMed PMID: 16794396; PubMed Central PMCID: PMCPMC1570598.
105. Cho Y B, Lee W Y, Choi S J, Kim J, Hong H K, Kim S H, et al. CC chemokine ligand 7 expression in liver metastasis of colorectal cancer. Oncol Rep. 2012; 28(2):689-94. doi: 10.3892/or.2012.1815. PubMed PMID: 22614322.
106. Nomiyama H, Hieshima K, Nakayama T, Sakaguchi T, Fujisawa R, Tanase S, et al. Human CC chemokine liver-expressed chemokine/CCL16 is a functional ligand for CCR1, CCR2 and CCR5, and constitutively expressed by hepatocytes. International immunology. 2001; 13(8):1021-9. PubMed PMID: 11470772.
107. Rubie C, Oliveira V, Kempf K, Wagner M, Tilton B, Rau B, et al. Involvement of chemokine receptor CCR6 in colorectal cancer metastasis. Tumour Biol. 2006; 27(3):166-74. doi: 10.1159/000092777. PubMed PMID: 16641550.
108. Brunner P M, Glitzner E, Reininger B, Klein I, Stary G, Mildner M, et al. CCL7 contributes to the TNF-alpha-dependent inflammation of lesional psoriatic skin. Experimental dermatology. 2015; 24(7):522-8. doi: 10.1111/exd.12709. PubMed PMID: 25828150.
109. Ferrari S M, Ruffilli I, Colaci M, Antonelli A, Ferri C, Fallahi P. CXCL10 in psoriasis. Adv Med Sci. 2015; 60(2):349-54. doi: 10.1016/j.advms.2015.07.011. PubMed PMID: 26318079.
110. Congjun J, Yanmei Z, Huiling J, Zhen Y, Shuo L. Elevated Local and Serum CX3CL1 (Fractalkine) Expression and Its Association with Disease Severity in Patients with Psoriasis. Ann Clin Lab Sci. 2015; 45(5):556-61. PubMed PMID: 26586708.
111. Hedrick M N, Lonsdorf A S, Hwang S T, Farber J M. CCR6 as a possible therapeutic target in psoriasis. Expert Opin Ther Targets. 2010; 14(9):911-22. doi: 10.1517/14728222.2010.504716. PubMed PMID: 20629596; PubMed Central PMCID: PMCPMC3700805.
112. Liu C, Cui G, Zhu M, Kang X, Guo H. Neuroinflammation in Alzheimer's disease: chemokines produced by astrocytes and chemokine receptors. Int J Clin Exp Pathol. 2014; 7(12):8342-55. PubMed PMID: 25674199; PubMed Central PMCID: PMCPMC4314046.
113. Nogueira L G, Santos R H, Ianni B M, Fiorelli Al, Mairena E C, Benvenuti L A, et al. Myocardial chemokine expression and intensity of myocarditis in Chagas cardiomyopathy are controlled by polymorphisms in CXCL9 and CXCL10. PLoS neglected tropical diseases. 2012; 6(10):e1867. doi: 10.1371/journal.pntd.0001867. PubMed PMID: 23150742; PubMed Central PMCID: PMC3493616.
114. Borst O, Schaub M, Walker B, Sauter M, Muenzer P, Gramlich M, et al. CXCL16 is a novel diagnostic marker and predictor of mortality in inflammatory cardiomyopathy and heart failure. International journal of cardiology. 2014; 176(3):896-903. Epub 2014 Sep. 17. doi: 10.1016/j.ijcard.2014.08.033. PubMed PMID: 25223819.
115. Escher F, Vetter R, Kuhl U, Westermann D, Schultheiss H P, Tschope C. Fractalkine in human inflammatory cardiomyopathy. Heart. 2011; 97(9):733-9. doi: 10.1136/hrt.2010.205716. PubMed PMID: 21357373.
116. Lassner D, Kuhl U, Siegismund C S, Rohde M, Elezkurtaj S, Escher F, et al. Improved diagnosis of idiopathic giant cell myocarditis and cardiac sarcoidosis by myocardial gene expression profiling. Eur Heart J. 2014; 35(32):2186-95. doi: 10.1093/eurheartj/ehu101. PubMed PMID: 24667923.
117. Kittleson M M, Minhas K M, Irizarry R A, Ye S Q, Edness G, Breton E, et al. Gene expression in giant cell myocarditis: Altered expression of immune response genes. International journal of cardiology. 2005; 102(2):333-40. doi: 10.1016/j.ijcard.2005.03.075. PubMed PMID: 15982506.
118. Fahmy N M, Yamani M H, Starling R C, Ratliff N B, Young J B, McCarthy P M, et al. Chemokine and receptor-gene expression during early and late acute rejection episodes in human cardiac allografts. Transplantation. 2003; 75(12):2044-7. doi: 10.1097/01.TP.0000069601.73079.94. PubMed PMID: 12829909.
119. Fuse K, Kodama M, Hanawa H, Okura Y, Ito M, Shiono T, et al. Enhanced expression and production of monocyte chemoattractant protein-1 in myocarditis. Clinical and experimental immunology. 2001; 124(3):346-52. PubMed PMID: 11472393; PubMed Central PMCID: PMCPMC1906075.
120. Lehmann M H, Kuhnert H, Muller S, Sigusch H H. Monocyte chemoattractant protein 1 (MCP-1) gene expression in dilated cardiomyopathy. Cytokine. 1998; 10(10):739-46. doi: 10.1006/cyto.1998.0354. PubMed PMID: 9811526.
121. Buch M H, Bingham S J, Bryer D, Emery P. Long-term infliximab treatment in rheumatoid arthritis: subsequent outcome of initial responders. Rheumatology (Oxford). 2007; 46(7):1153-6. doi: 10.1093/rheumatology/kem075. PubMed PMID: 17478470.
122. McInnes I B, Schett G. The pathogenesis of rheumatoid arthritis. The New England journal of medicine. 2011; 365(23):2205-19. doi: 10.1056/NEJMra1004965 10.7748/phc2011.11.21.9.29.c8797. PubMed PMID: 22150039.
123. Feagan B G, Rutgeerts P, Sands B E, Hanauer S, Colombel J F, Sandborn W J, et al. Vedolizumab as induction and maintenance therapy for ulcerative colitis. The New England journal of medicine. 2013; 369(8):699-710. doi: 10.1056/NEJMoa1215734. PubMed PMID: 23964932.
124. Chao G, Lau W L, Hackel B J, Sazinsky S L, Lippow S M, Wittrup K D. Isolating and engineering human antibodies using yeast surface display. Nat Protoc. 2006; 1(2):755-68.
125. Deruaz M, Bonvin P, Severin I C, Johnson Z, Krohn S, Power C A, et al. Evasin-4, a tick-derived chemokine-binding protein with broad selectivity can be modified for use in preclinical disease models. FEBS Journal. 2013; 280(19):4876-87.
126. Concepcion J, Witte K, Wartchow C, Choo S, Yao D, Persson H, et al. Label-free detection of biomolecular interactions using BioLayer interferometry for kinetic characterization. Comb Chem High Throughput Screen. 2009; 12(8):791-800.
127. Zuern C S, Walker B, Sauter M, Schaub M, Chatterjee M, Mueller K, et al. Endomyocardial expression of SDF-1 predicts mortality in patients with suspected myocarditis. Clin Res Cardiol. 2015; 104(12):1033-43.
128. Orn S, Breland U M, Mollnes T E, Manhenke C, Dickstein K, Aukrust P, et al. The chemokine network in relation to infarct size and left ventricular remodeling following acute myocardial infarction. Am J Cardiol. 2009; 104(9):1179-83.
129. Eardley K S, Smith S W, Cockwell P. Chemokines in vasculitis. Front Biosci (Elite Ed). 2009; 1:26-35.
130. Brix S R, Stege G, Disteldorf E, Hoxha E, Krebs C, Krohn S, et al. CC Chemokine Ligand 18 in ANCA-Associated Crescentic G N. J Am Soc Nephrol. 2015; 26(9):2105-17.
131. Dallos T, Heiland G R, Strehl J, Karonitsch T, Gross W L, Moosig F, et al. CCL17/thymus and activation-related chemokine in Churg-Strauss syndrome. Arthritis and rheumatism. 2010; 62(11):3496-503.
132. Eriksson P, Andersson C, Cassel P, Nystrom S, Ernerudh J. Increase in Th17-associated CCL20 and decrease in Th2-associated CCL22 plasma chemokines in active ANCA-associated vasculitis. Scandinavian journal of rheumatology. 2015; 44(1):80-3.
133. Blaschke S, Brandt P, Wessels J T, Muller G A. Expression and function of the C-class chemokine lymphotactin (XCL1) in Wegener's granulomatosis. J Rheumatol. 2009; 36(11):2491-500.
134. Bruhl H. Expression of DARC, CXCR3 and CCR5 in giant cell arteritis. Rheumatology. 2005; 44(3):309-13.
135. Deng J, Ma-Krupa W, Gewirtz A T, Younge B R, Goronzy J J, Weyand C M. Toll-like receptors 4 and 5 induce distinct types of vasculitis. Circ Res. 2009; 104(4):488-95.
136. Corbera-Bellalta M, Planas-Rigol E, Lozano E, Terrades-Garcia N, Alba M A, Prieto-Gonzalez S, et al. Blocking interferon γ reduces expression of chemokines CXCL9, CXCL10 and CXCL11 and decreases macrophage infiltration in ex vivo cultured arteries from patients with giant cell arteritis. Annals of the rheumatic diseases. 2016; 75(6):1177-86.
137. Dhawan V, Mahajan N, Jain S. Role of C-C chemokines in Takayasu's arteritis disease. International journal of cardiology. 2006; 112(1):105-11.
138. Noris M, Daina E, Gamba S, Bonazzola S, Remuzzi G. Interleukin-6 and RANTES in Takayasu arteritis: a guide for therapeutic decisions? Circulation. 1999; 100(1):55-60.
139. Mirabelli-Badenier M, Braunersreuther V, Viviani G L, Dallegri F, Quercioli A, Veneselli E, et al. CC and CXC chemokines are pivotal mediators of cerebral injury in ischaemic stroke. Thrombosis and haemostasis. 2011; 105(3):409-20.
140. Czaja A J. Review article: chemokines as orchestrators of autoimmune hepatitis and potential therapeutic targets. Alimentary pharmacology &t therapeutics. 2014; 40(3):261-79.
141. Sasaki M, Miyakoshi M, Sato Y, Nakanuma Y. Chemokine-chemokine receptor CCL2-CCR2 and CX3CL1-CX3CR1 axis may play a role in the aggravated inflammation in primary biliary cirrhosis. Dig Dis Sci. 2014; 59(2):358-64.
142. Dyson J K, Hirschfield G M, Adams D H, Beuers U, Mann D A, Lindor K D, et al. Novel therapeutic targets in primary biliary cirrhosis. Nat Rev Gastroenterol Hepatol. 2015; 12(3):147-58.
143. Landi A, Weismuller T J, Lankisch T O, Santer D M, Tyrrell D L, Manns M P, et al. Differential serum levels of eosinophilic eotaxins in primary sclerosing cholangitis, primary biliary cirrhosis, and autoimmune hepatitis. J Interferon Cytokine Res. 2014; 34(3):204-14.
144. Borchers A T, Shimoda S, Bowlus C, Keen C L, Gershwin M E. Lymphocyte recruitment and homing to the liver in primary biliary cirrhosis and primary sclerosing cholangitis. Semin Immunopathol. 2009; 31(3):309-22.
145. Hirschfield G M, Gershwin M E. The immunobiology and pathophysiology of primary biliary cirrhosis. Annu Rev Pathol. 2013; 8:303-30.
146. Li Y, Wang W, Tang L, He X, Yan X, Zhang X, et al. Chemokine (C-X-C motif) ligand 13 promotes intrahepatic chemokine (C-X-C motif) receptor 5+ lymphocyte homing and aberrant B-cell immune responses in primary biliary cirrhosis. Hepatology. 2015; 61(6):1998-2007.
147. Heydtmann M, Lalor P F, Eksteen J A, Hubscher S G, Briskin M, Adams D H. CXC Chemokine Ligand 16 Promotes Integrin-Mediated Adhesion of Liver-Infiltrating Lymphocytes to Cholangiocytes and Hepatocytes within the Inflamed Human Liver. The Journal of Immunology. 2005; 174(2):1055-62.
148. Eksteen B, Grant A J, Miles A, Curbishley S M, Lalor P F, Hubscher S G, et al. Hepatic endothelial CCL25 mediates the recruitment of CCR9+ gut-homing lymphocytes to the liver in primary sclerosing cholangitis. J Exp Med. 2004; 200(11):1511-7.
149. Sahin H, Wasmuth H E. Chemokines in tissue fibrosis. Biochim Biophys Acta. 2013; 1832(7):1041-8.
150. Xu Z, Zhang X, Lau J, Yu J. C-X-C motif chemokine 10 in non-alcoholic steatohepatitis: role as a pro-inflammatory factor and clinical implication. Expert Rev Mol Med. 2016; 18:e16.
151. Krenkel O, Mossanen J C, Tacke F. Immune mechanisms in acetaminophen-induced acute liver failure. Hepatobiliary Surg Nutr. 2014; 3(6):331-43.
152. Marra F, Tacke F. Roles for chemokines in liver disease. Gastroenterology. 2014; 147(3):577-94 el.
153. Hartl D, Griese M, Nicolai T, Zissel G, Prell C, Reinhardt D, et al. A role for MCP-1/CCR2 in interstitial lung disease in children. Respir Res. 2005; 6:93.
154. Schmidt K, Martinez-Gamboa L, Meier S, Witt C, Meisel C, Hanitsch L G, et al. Bronchoalveoloar lavage fluid cytokines and chemokines as markers and predictors for the outcome of interstitial lung disease in systemic sclerosis patients. Arthritis research &t therapy. 2009; 11(4):R111.
155. Capelli A, Di Stefano A, Gnemmi I, Donner C F. CCR5 expression and CC chemokine levels in idiopathic pulmonary fibrosis. Eur Respir J. 2005; 25(4):701-7.
156. Willems S, Verleden S E, Vanaudenaerde B M, Wynants M, Dooms C, Yserbyt J, et al. Multiplex protein profiling of bronchoalveolar lavage in idiopathic pulmonary fibrosis and hypersensitivity pneumonitis. Ann Thorac Med. 2013; 8(1):38-45.
157. DePianto D J, Chandriani S, Abbas A R, Jia G, N'Diaye E N, Caplazi P, et al. Heterogeneous gene expression signatures correspond to distinct lung pathologies and biomarkers of disease severity in idiopathic pulmonary fibrosis. Thorax. 2015; 70(1):48-56.
158. Schupp J C, Binder H, Jager B, Cillis G, Zissel G, Muller-Quernheim J, et al. Macrophage activation in acute exacerbation of idiopathic pulmonary fibrosis. PLoS One. 2015; 10(1):e0116775.
159. Petrek M, Pantelidis P, Southcott A M, Lympany P, Safranek P, Black C M, et al. The source and role of RANTES in interstitial lung disease. Eur Respir J. 1997; 10(6):1207-16.
160. Belperio J A, Dy M, Murray L, Burdick M D, Xue Y Y, Strieter R M, et al. The Role of the Th2 CC Chemokine Ligand CCL17 in Pulmonary Fibrosis. The Journal of Immunology. 2004; 173(7):4692-8.
161. Antoniou K M, Tzouvelekis A, Alexandrakis M G, Sfiridaki K, Tsiligianni I, Rachiotis G, et al. Different angiogenic activity in pulmonary sarcoidosis and idiopathic pulmonary fibrosis. Chest. 2006; 130(4):982-8.
162. Vasakova M, Sterclova M, Kolesar L, Slavcev A, Pohunek P, Sulc J, et al. Bronchoalveolar lavage fluid cellular characteristics, functional parameters and cytokine and chemokine levels in interstitial lung diseases. Scand J Immunol. 2009; 69(3):268-74.
163. Vuga L J, Tedrow J R, Pandit K V, Tan J, Kass D J, Xue J, et al. C-X-C motif chemokine 13 (CXCL13) is a prognostic biomarker of idiopathic pulmonary fibrosis. Am J Respir Crit Care Med. 2014; 189(8):966-74.
164. Haringman J J, Smeets T J, Reinders-Blankert P, Tak P P. Chemokine and chemokine receptor expression in paired peripheral blood mononuclear cells and synovial tissue of patients with rheumatoid arthritis, osteoarthritis, and reactive arthritis. Annals of the rheumatic diseases. 2006; 65(3):294-300.
165. Hijnen D, De Bruin-Weller M, Oosting B, Lebre C, De Jong E, Bruijnzeel-Koomen C, et al. Serum thymus and activation-regulated chemokine (TARC) and cutaneous T cell-attracting chemokine (CTACK) levels in allergic diseases: TARC and CTACK are disease-specific markers for atopic dermatitis. The Journal of allergy and clinical immunology. 2004; 113(2):334-40.
166. Echigo T, Hasegawa M, Shimada Y, Takehara K, Sato S. Expression of fractalkine and its receptor, CX3CR1, in atopic dermatitis: possible contribution to skin inflammation. The Journal of allergy and clinical immunology. 2004; 113(5):940-8.

1.-28. (canceled)

29. A composition comprising:
(I) a polypeptide comprising (a) all or part of an amino acid sequence shown in any one of SEQ ID NOs: 1 to 31: or (b) all or part of an amino acid sequence characterized by at least 70% homology or amino identity to a sequence of (a) over its entire length; or
(II) a fusion polypeptide comprising a polypeptide according to (I) linked to a second peptide or polypeptide; or
(III) a combination of two or more polypeptides according to (I) or (II).

30. The composition of matter according to claim 29(I), wherein said polypeptide comprises (a) all or part of an amino acid sequence shown in any one of SEQ ID NOs: 1-3, 6-10, 15-16, 20-23, or 29-30; or (b) all or part of an amino acid sequence characterized by at least 70% homology or amino identity to a sequence of (a) over its entire length, wherein said polypeptide binds one or more human chemokines selected from CCL2, CCL13, and/or CCL20.

31. The composition of matter according to claim 30, wherein said polypeptide comprises (a) all or part of an amino acid sequence shown in any one of SEQ ID NOs: 1, 9, or 29; or (b) all or part of an amino acid sequence characterized by at least 70% homology or amino identity to a sequence of (a) over its entire length.

32. The composition of matter according to claim 29(I), wherein said polypeptide comprises (a) all or part of an amino acid sequence shown in any one of SEQ ID NOs: 5, 19, 24-26, 28, or 31; or (b) all or part of an amino acid sequence characterized by at least 70% homology or amino identity to a sequence of (a) over its entire length, wherein said polypeptide binds one or more human chemokines selected from CXCL3, CXCL10, and/or CXCL12.

33. The composition of matter according to claim 29(II), wherein the second polypeptide comprised in the fusion polypeptide is a fragment crystallisable region (Fc region).

34. A method comprising:
(A) producing a polypeptide according to claim 29(I) or a combination according to claim 29(III) comprising culturing a host cell comprising a polynucleotide, a combination of polynucleotides or a vector that encodes a polypeptide according to 29(I) or a combination according to 29(III) under conditions which produce the polypeptide or the combination; o
(B) inhibiting the signalling of one or more chemokines in an in vitro culture, the method comprising contacting the culture with a polypeptide according to claim 29(I), or a combination according to claim 29(III).

35. A method of inhibiting the signalling of one or more chemokines in a subject, the method comprising administering to the subject: (I) a polypeptide comprising (a) all or part of an amino acid sequence shown in any one of SEQ ID NOs: 1 to 31; or (b) all or part of an amino acid sequence characterized by at least 70% homology or amino identity to a sequence of (a) over its entire length, (II) a fusion polypeptide comprising a polypeptide according to (I) linked to a second peptide or polypeptide or (III) a combination of two or more of said polypeptides according to (I) or (II).

36. A method of treating or preventing in a subject one or more diseases associated with one or more chemokines, the method comprising administering to the subject a polypeptide: (I) comprising (a) all or part of an amino acid sequence shown in any one of SEQ ID NOs: 1 to 31; or (b) all or part of an amino acid sequence characterized by at least 70% homology or amino identity to a sequence of (a) over its entire length, (II) a fusion polypeptide comprising a polypeptide according to (I) linked to a second peptide or polypeptide, or (III) a combination of two or more of said polypeptides according to (I) or (II).

37. The method according to claim 36, wherein the polypeptide or the combination is administered in combination with another therapy.

38. The method according to claim 36, wherein the one or more chemokines are one or more of CCL1, CCL2, CCL3, CCL4, CCL5, CCL8, CCL11, CCL15, CCL17, CCL18, CCL19, CCL20, CCL22, CCL25, CX3CL1, CXCL8, CXCL10, CXCL11, or CXCL12.

39. The method according to claim 36, wherein the one or more diseases are one or more of alcohol liver injury, Alzheimer's disease, ANCA vasculitis, atherosclerosis, atopic dermatitis, autoimmune hepatitis, breast cancer, Chagas myocarditis, colorectal cancer, giant cell arteritis, giant cell myocarditis, heart allograft rejection, idiopathic pulmonary fibrosis, inflammatory bowel disease, kidney fibrosis, liver fibrosis, lung fibrosis, multiple sclerosis, myocardial infarction, myocarditis, myocarditis enteroviral, non alcoholic steatohepatitis, paracetamol liver injury, primary biliary cirrhosis, primary sclerosing cholangitis, psoriasis, rheumatoid arthritis, skin fibrosis, stroke, or Takayasu disease.

40. The method according to claim 36, wherein the one or more diseases associated with the one or more chemokines is an inflammatory disease comprising expression of more than one chemokine.

41. The method according to claim 40, wherein said disease is myocarditis, giant cell myocarditis, myocardial infarction, stroke, or idiopathic pulmonary fibrosis.

42. The method according to claim 36, wherein the polypeptide according to (I) comprises (a) all or part of an amino acid sequence shown in any one of SEQ ID NOs: 1, 9, or 29; or (b) all or part of an amino acid sequence having at least 70% homology or amino identity to a sequence of (a) over its entire length, or a corresponding combination according to (III).

43. A method of detecting one or more chemokines in a tissue, comprising contacting the tissue with a detectably-labelled polypeptide according to claim 29(I) or a detectably-labelled combination according to claim 29(III) and detecting the binding of the polypeptide or the combination to one or more chemokines.

44. The method according to claim 43, wherein the one or more chemokines are one or more of CCL1, CCL2, CCL3, CCL4, CCL5, CCL8, CCL11, CCL15, CCL17, CCL18, CCL19, CCL20, CCL22, CCL25, CX3CL1, CXCL8, CXCL10, CXCL11, or CXCL12.

45. The method according to claim 43, wherein the method is for diagnosing or prognosing one or more diseases selected from one or more of alcohol liver injury, Alzheimer's disease, ANCA vasculitis, atherosclerosis, atopic dermatitis, autoimmune hepatitis, breast cancer, Chagas myocarditis, colorectal cancer, giant cell arteritis, giant cell myocarditis, heart allograft rejection, idiopathic pulmonary fibrosis, inflammatory bowel disease, kidney fibrosis, liver fibrosis, lung fibrosis, multiple sclerosis, myocardial infarction, myocarditis, myocarditis enteroviral, non alcoholic steatohepatitis, paracetamol liver injury, primary biliary cirrhosis, primary sclerosing cholangitis, psoriasis, rheumatoid arthritis, skin fibrosis, stroke, or Takayasu disease.
